(19) United States
(12) Patent Application Publication
(10) Pub. No.: US 2024/0252027 A1
(43) Pub. Date: Aug. 1, 2024

(54) ENDOSCOPE WITH AN INSERTION CORD INCLUDING AN INTERSTITIAL LUMEN

(71) Applicant: AMBU A/S, Ballerup (DK)

(72) Inventor: Morten SØRENSEN, Ballerup (DK)

(73) Assignee: AMBU A/S, Ballerup (DK)

(21) Appl. No.: 18/289,610

(22) PCT Filed: May 20, 2022
(86) PCT No.: PCT/EP2022/063812

§ 371 Date: Nov. 6, 2023

(30) Foreign Application Priority Data
May 21, 2021 (DK) PA 2021 70265
May 21, 2021 (DK) PA 2021 70266

(51) Int. Cl. A61B 1/015; A61B 1/00; A61B 1/005; A61B 1/018; A61B 1/05

(52) U.S. Cl. CPC A61B 1/015; A61B 1/00009; A61B 1/0011; A61B 1/0052; A61B 1/0055; A61B 1/0057; A61B 1/018; A61B 1/05

(57) ABSTRACT

An endoscope (1) includes a handle (2) including a handle housing (2a), an irrigation inlet port (46) adapted to receive irrigation fluid, and an irrigation channel (42); and an insertion cord (10) extending distally from the handle. The insertion cord includes an insertion tube (11); a bending section (12) extending from the insertion tube; a distal tip (13) extending from the bending section and including a tip housing; a camera at least partially enclosed in the tip housing; interstitial space (26) within the insertion tube in fluid communication with the irrigation channel; and at least one interstitial flow opening (64-67) in fluid communication with the interstitial space and adapted to discharge the irrigation fluid.

### CROSS REFERENCE TO RELATED APPLICATIONS

This application claims priority from and the benefit of Danish Patent Applications Nos. PA 2021 70265, filed May 21, 2021, and PA 2021 70266, filed May 21, 2021; the contents of the aforementioned applications are incorporated herein by reference in their entirety.

### TECHNICAL FIELD

The disclosure relates to endoscopes and, in particular, to endoscopes with irrigation channels.

### BACKGROUND

Generally, an endoscope includes a handle and a shaft which is inserted into the body to a target site. A working channel may be provided through which various tools may be inserted to reach the target site. A ureteroscope is a type of endoscope used to examine a patient's kidneys. In a typical procedure, laser lithotripsy is performed to break stones into multiple smaller fragments which are collected within the calyx. Each fragment is then individually removed using a device inserted through the working channel of the ureteroscope. In another procedure, a vacuum system is used in which the stone fragments are sucked out through the working channel. During this procedure, irrigation fluid is pumped into the kidney through an irrigation channel or the working channel and is expirated out, with the fragmented stones, through the working channel.

U.S. Patent Application No. 2020/0196843 discloses a ureteroscope including a first port and a second port and a shaft including a shaft lumen open at the distal end of the shaft. The first port is in communication with a handle channel which is in communication with the shaft lumen and the second port is in communication with the working channel extending through the handle and the shaft lumen to the distal end of the shaft. Hubs are connected to the shaft lumen and the working channel to seal them so that no fluid can enter into the handle cavity through the hubs. An end cap at the distal end of the shaft includes a plurality of irrigation holes fluidly coupled to the shaft lumen and provided to irrigate the target site.

U.S. Pat. No. 8,784,298 discloses an endoscope instrument including an insertion tube with a distal portion presenting an outlet section and housing an appliance (a tubular duct) for occupying a retracted position inside the tube and a working position in which the appliance occupies at least a portion of the outlet section. The distal portion of the insertion tube includes at least one radially deformable wall over a portion of its length extending from the outlet section, enabling the outlet section of the distal portion of the tube to be increased on the appliance passing from its retracted position (Abstract). Irrigation may be fed through the appliance (col. 5, I. 12-17).

U.S. Pat. No. 10,492,662 discloses integrated endoscope irrigation in the form of an endoscopic device in which the empty spaces between the plurality of tubes, wires, and cables are utilized as a channel that enables liquid or gas to flow from a handle section to a distal tip. The distal tip comprises a pattern of alternating grooves and lands on its outer surface and a cap that fits tightly over the lands extends to and slightly beyond the far end of the distal tip. The tight fitting cap changes the grooves into closed channels through which liquid or gas can flow to an annular nozzle provided by a gap between the front face of the distal tip and a bottom surface of an annular curved portion of the cap

U.S. Patent Publication No. 2017/0215965 discloses a medical device, a ureteroscope, providing for use of a laser to break kidney stones into smaller fragments or dust for removal from a patient. The ureteroscope comprises a tube with two or, in an embodiment shown, three lumens. At the distal end the ureteroscope comprises a distal portion or cap with three openings at the far end corresponding to the three lumens. A first lumen/opening provides for introduction of a laser fiber and/or flow of irrigation fluid, a second lumen/opening provides for suction, and a third lumen provides for wires/fibers/etc. necessary for controlling and communication with an imaging and/or illumination device(s) located at a distal end of the medical device. In some examples, a fourth and/or fifth lumen may be included for, e.g., a separate lumen exclusively for the introduction of the laser fiber or an additional lumen for the introduction of fluid through side ports. The lumens included in the tube may be any size, shape, and/or in any configuration. The first lumen may fork and/or include a plurality of small branches at multiple locations to place first lumen in fluid communication with a plurality of side ports to allow the flow of irrigation fluid within first lumen to exit into the kidney at a plurality of locations along an exterior circumference of distal portion. It is further suggested to position side ports on the sides of tube, whereby more surface area will be available on the distal face of tube (e.g., by minimizing the volume of fluid through the distal end of first lumen and thus cross-sectional area of first lumen at the distal end). The cap may be integral with a sheath which fits along a portion of the tube or a sheath that extends substantially the entire length of the endoscope.

Commonly-owned International Application No. PCT/US2020/030605, published as WO2020/0223429A1, discloses a ureteroscope with a fluid port at the handle for introducing irrigation fluid into a space between the outer wall of the shaft and the working channel within the shaft, denoted as the interstitial space. A fluid seal is provided at the handle, between the fluid port and the outer diameter of the working channel. Pull wires and other wires can pass through the fluid seal, which prevents fluid in the interstitial space in the shaft from flowing into the handle. Some elements passing through fluid seal, such a camera wire, may not need to translate and thus are glued/sealed into place. At a tip at the distal end of the shaft is an opening configured to discharge the irrigation fluid to help clear away debris from the field of view of the camera.

Improvements are desirable to further reduce the size of endoscopes, including ureteroscopes, while simultaneously increasing their functionality.

### SUMMARY

It is an object of the present disclosure to provide solutions which overcome or reduce at least some of the limitations of the prior art visualization devices. One solution is a visualization device with a reduced distal end cross-section, said reduction being relative to prior art devices performing the same functions. Another solution is a method of making said visualization device, said method being lower-cost relative to prior art devices performing the same functions. Another solution is a system including a video processing apparatus and said visualization device.

The disclosure also relates to small diameter endoscopes where both water irrigation and suction is needed.

It is an object of the present disclosure to provide a construction of an insertion cord for an endoscope that facilitates construction with a small diameter of the insertion cord and especially of a distal end thereof.

In accordance with the aforementioned object and solutions, the present disclosure provides a visualisation device, such as an endoscope, more particularly a ureteroscope, with an interstitial lumen provided to irrigate a distal end of the device. The present disclosure further provides a visualization device with an outer sleeve, or tubular sheath, disposed over a bending section of an insertion cord of the visualization device, which provides at least one bending section fluid opening to permit irrigation of the space surrounding the distal end of insertion cord. The bending section fluid opening may be supplied irrigation fluid via the interstitial lumen or via any other lumen or channel. The interstitial lumen may provide irrigation liquid to the bending section fluid opening or to any other opening at the distal end of the device.

In a first aspect of the present disclosure, an interstitial lumen is provided in an insertion cord of a visualization device, such as an endoscope, the interstitial lumen flowing irrigation liquid to the distal end of the insertion cord. Some embodiments of visualization devices according to the first aspect are described immediately below.

In some embodiments, said endoscope includes a seal between the interstitial lumen and an internal space of a position interface. In some examples, the position interface is a handle comprising a handle housing having an internal space. The seal may be positioned, at least partly, in the internal space.

In some embodiments, said endoscope is provided with an outer sleeve, or tubular sheath, disposed over a bending section of the insertion cord. The outer sleeve provides at least one fluid opening to permit irrigation of the space surrounding the distal end of insertion cord.

In some variations of the present embodiment, the at least one fluid opening is closed in a relaxed condition of the outer tubular sheath and opens when the outer tubular sheath is subject to a pressure of fluid introduced through the proximal end of the insertion cord. Hereby a valve function is obtained that prevents or limits back reflux of fluid into the interstitial lumen.

In some variations of the present embodiment, the outer tubular sheath is of a stretchable material adapted to be stretched when subject to a pressure of fluid introduced through the proximal end of the insertion cord. Hereby is obtained that stretching the material of the tubular sheath may allow the at least one opening to open and relaxation of said pressure may result in the stretchable material returning to its pre-stretched condition closing the at least one opening.

In some variations of the present embodiment, a distal portion of the outer tubular sheath is externally surrounding a part of the elongated body part and is preferably attached to the circumferential body surface thereof at separate positions circumferentially spaced around said circumferential body surface, thus leaving the outer tubular sheath unattached to the circumferential body surface at at least one position between spaced positions of attachment. Hereby may e.g. be obtained that irrigation fluid may leak out along the surface of the elongated body part in a direction towards the area in front of the distal end of the insertion cord. At the same time the attachments prevent displacement of the tubular sheath during insertion of the endoscope insertion cord.

In some variations of the present embodiment, the outer tubular sheath, at least in a relaxed condition, surrounds the circumferential body surface with a tight fit. Hereby a valve function as mentioned above may be obtained.

In some variations of the present embodiment, the elongated body part comprises at least one groove in the circumferential body surface, said at least one groove extending from a proximal end thereof to a distal end thereof, and said groove extending between positions of attachment of the outer tubular sheath to the circumferential body surface. Hereby the flow of irrigation fluid out of the interstitial lumen along the circumferential surface may e.g. be facilitated and focussed by an at least one channel provided by the at least one groove.

In some variations of the present embodiment, the at least one fluid opening is provided by a slit through the material of the outer tubular sheath. Such slit may provide an opening that is closed when the material of the outer tubular sheath is in a relaxed condition and that opens when subject to an internal pressure of fluid.

In some embodiments, said endoscope includes the aforementioned interstitial lumen, and the at least one fluid opening is fluidly coupled to the interstitial lumen to permit irrigation through the at least one fluid opening.

In some embodiments, said sealing body comprises at least one integral part moulded in one piece, said integral part comprising a first portion with at least a first part extending snugly along at least a part of a circumference of at least one of any second tubular body; wire; and wire pipe and a second portion abutting snugly towards the first tubular body, and wherein a fluid passage for communicating a fluid with the interstitial lumen is attached to the sealing body. In an embodiment a construction is obtained that facilitates production, e.g. when the endoscope is produced as a single use, disposable object.

By the expression the “second portion abutting snugly towards the first tubular body” should be understood that the second portion provides for sealing relative to the first tubular body, either directly by contact with the first tubular body, possibly with an intermediate layer of sealant, such as glue, or indirectly through another element.

The endoscope provides for application of different tools through the working channel. In case of use as a ureteroscope the tool may comprise an optic fiber for applying laser pulses e.g. to a kidney stone inside a patient to break or dissolve the kidney stone for subsequent removal by suction through the insertion cord, e.g. through the working channel.

In an embodiment the second portion comprises a second part extending snugly along at least a part of a circumference of the first tubular body. Hereby a need for further sealing between the first tubular body and the internal space of the handle housing may be avoided.

In some embodiments the sealing body comprises a number of two or more of said integral parts, and the first parts of the first portions of said integral parts jointly extend snugly around the entire circumferences of the at least one of any second tubular body; wire; and wire pipe. In other embodiments the sealing body comprises a number of two or more of said integral parts, whereby the first parts of the first portions of said integral parts jointly extend snugly around the entire circumferences of the at least one of any second tubular body; wire; and wire pipe, and whereby the second parts of the second portions of said integral parts jointly extend snugly around the entire circumferences of the first tubular body. Such embodiments facilitate production of the sealing body as moulded parts ready for subsequent assembly.

In a further embodiment the sealing body comprises a first sealing body portion comprising the joint first portions of the integral parts and a second sealing body portion comprising the joint second portions of the integral parts, a first direction is extending from the first sealing body portion towards the second sealing body portion, and the first sealing body portion is extended to have a larger extent relative to an extent the second sealing body portion in a second direction perpendicular to the first direction along a dividing line between said integral parts, the at least one of any second tubular body; wire; wire pipe; and fluid passage being positioned on said dividing line.

In a further embodiment more of said at least one of any second tubular body; wire; wire pipe; and fluid passage are positioned on said dividing line and are mutually spaced whereby the integral parts on either side of the dividing line meet on the dividing line between any pair of said at least one of any second tubular body; wire; wire pipe; and fluid passage for the first parts of said integral parts jointly to extend snugly around each of said at least one of any second tubular body; wire; wire pipe; and fluid passage individually.

Further, in a such embodiment the first sealing body portion is extended to have a larger extent in the second direction relative to the extent of the first sealing body portion in a third direction perpendicular to the first and the second direction.

In some embodiments the fluid passage comprises a tubular element, and third parts of the first portions of said integral parts jointly extend snugly around an entire circumferences of the tubular element. Hereby is facilitated use of an internal tube or hose inside the handle housing for connecting a source of irrigation liquid.

In an embodiment the sealing body is constituted by a single moulded body. In a further embodiment the single moulded body is moulded in situ to seal in relation to first tubular body; and the at least one of any second tubular body; wires; wire pipes; and the fluid passage. Hereby is provided for moulding in situ of the sealing body, e.g. during assembly of the endoscope.

In an embodiment at least one wire is constituted by a Bowden cable comprising an inner cable, typically called a steering wire or pull wire housed in a hollow outer cable housing, typically called a wire pipe, whereby the hollow outer cable housing comprises a pipe sealing extending from the first part and a distance in at least one of a distal direction and a proximal direction of the Bowden cable. The hollow outer cable housing of a Bowden cable is often made from a coiled metal wire. However, such a coiled metal wire will not be watertight, and therefor water in the interior space of the insertion cord may leak through the wire pipes and into the interior space of the handle. By this embodiment sealing of the Bowden cable may be provided.

In an embodiment an inner space is defined inside the sealing body, said inner space being in fluid connection with the interstitial lumen and sealed relative to the internal space of the handle housing.

In a further embodiment a sealing glue is present in the hollow outer cable housing at the place where the hollow outer cable housing is passing the first part and a distance in at least one of the distal direction and the proximal direction of the Bowden cable, and wherein a slip means, such as oil is present between the sealing glue and the inner cable. Hereby is obtained sealing of the Bowden cable in case the pipe sealing does not extent a sufficient distance from the first portion of the sealing body to ensure proper sealing of the Bowden cable while it is still possible to pull the inner cable back and forth.

In a further embodiment the pipe sealing is constituted by the sealing glue. Thus the glue will in this embodiment basically penetrate the hollow outer cable housing to seal the same thereby making a separate pipe sealing superfluous.

In an embodiment the handle housing comprises a connector for connecting a fluid connection, especially a liquid connection, such as a hose, the handle housing further comprising two housing parts interconnected i.a. along respective channel interfaces extending from the connector to the sealing body at a point of attachment of the insertion cord to the handle housing, said channel interfaces providing between them a tubular channel extending along a part of the handle housing. Thus the need for an internal tube or hose inside the handle housing may be reduced or avoided. It should be understood, that the two housing parts may be interconnected along connection interfaces of the two housing parts wherein the channel interfaces constitute parts of said connection interfaces.

In general, the fluid passage for communicating a fluid with the interstitial lumen may be an irrigation liquid inlet for inlet of irrigation fluid to the interstitial lumen. As an alternative, the fluid passage may be used to applying suction to the interstitial lumen.

In some embodiments the endoscope comprises a handle housing and an insertion cord, the insertion cord comprising a first tubular body and having a proximal end and a distal end, the first tubular body housing at least one of: a working channel, constituted by a second tubular body; a wire; and a wire pipe, the first tubular body comprising an interstitial lumen between any present second tubular body; wire; and wire pipe, said interstitial lumen providing a channel for flowing a fluid through the insertion cord, the handle housing comprising an internal space, wherein the proximal end of the insertion cord is attached to the handle housing, and the interstitial lumen of the first tubular body is sealed relative to the internal space of the handle housing by a sealing body, wherein said sealing body comprises at least one integral part moulded in one piece, said integral part comprising a first portion with at least a first part extending snugly along at least a part of a circumference of at least one of any second tubular body; wire; and wire pipe and a second portion abutting snugly towards the first tubular body, and wherein a fluid passage for communicating a fluid with the interstitial lumen is attached to the sealing body.

In some embodiments the endoscope comprises a handle housing and an insertion cord, the insertion cord comprising a first tubular body and having a proximal end and a distal end, the first tubular body housing at least one of: a working channel, constituted by a second tubular body; a wire; and a wire pipe, the first tubular body comprising an interstitial lumen between any present second tubular body; wire; and wire pipe, said interstitial lumen providing a channel for flowing a fluid through the insertion cord, the handle housing comprising an internal space, wherein the proximal end of the insertion cord is attached to the handle housing, and the interstitial lumen of the first tubular body is sealed relative to the internal space of the handle housing by a sealing body, wherein said sealing body comprising a first body part and a second body part, each of said body parts comprising a first portion with at least a first part extending along at least a part of a circumference of at least one of any second tubular body; wire; and wire pipe and a second portion for sealing connection towards the first tubular body, and wherein a fluid passage for communicating a fluid with the interstitial lumen is attached to the sealing body. In an embodiment the first and the second body parts each comprises an assembly face for connection with other of the second and the first body part, and the first parts of the first portions of the first and second body part provide together in an assembled condition of the sealing body, a channel for the at least one of: a working channel, constituted by a second tubular body; a wire; and a wire pipe to extend into the sealing body.

In a variation of the present embodiment, the second portions of the first and second body part provide together in an assembled condition of the sealing body, a second channel for the first tubular body to extend into the sealing body. In an embodiment portions of the first and second body part provide together in an assembled condition of the sealing body, a third channel for the fluid passage to extend into the sealing body. In an embodiment, the assembly faces are provided with mutually corresponding ridges and valleys for securing mutual positioning in the assembled condition.

In an example of the present embodiment, at least one of the first, second, and third channel comprises an intermediate branch channel extending to an outer surface of the sealing body.

In some embodiments an endoscope, such as a ureteroscope, comprises a handle housing and an insertion cord, the insertion cord comprising a first tubular body and having a proximal end and a distal end, the first tubular body housing at least one of: a working channel, constituted by a second tubular body; a wire; and a wire pipe, the first tubular body comprising an interstitial lumen between any present second tubular body; wire; and wire pipe, said interstitial lumen providing a channel for flowing a fluid through the insertion cord, the handle housing comprising an internal space, wherein the proximal end of the insertion cord is attached to the handle housing, and the interstitial lumen of the first tubular body is sealed relative to the internal space of the handle housing by a sealing body.

In a second aspect, the present disclosure provides a visualization system comprising a visualization device according to the first aspect, and a video processing device, such as a monitor.

In a third aspect, a method of irrigating a space within a patient is provided, the method comprising: inserting the insertion cord of the endoscope according to the first aspect into the patient; supplying irrigation fluid to the irrigation channel of the endoscope, the irrigation fluid flowing through the interstitial space and discharging through an opening to the environment. The opening may be an interstitial flow opening according to the fifth aspect of the disclosure.

In a fourth aspect, the present disclosure provides an endoscope in which the insertion cord comprises a bending section and an outer tubular sheath providing an external surface of at least a portion of the bending section. Said outer tubular sheath provides at least one fluid opening for fluid to pass from the bending section to a surrounding of the insertion cord. Some embodiments of endoscopes according to the third aspect are described immediately below.

Providing irrigation fluid through the at least one fluid opening provided by the outer tubular sheath voids the need to provide an irrigation through the distal tip of the insertion cord, thus the overall diameter of the distal tip, and of at least the bending section, may be reduced.

In some embodiments, the insertion cord includes an interstitial lumen provided to flow irrigation liquid to the at least one fluid opening.

In some embodiments, an endoscope comprises a first tubular body and having a proximal end and a distal end, the first tubular body housing at least one of: a second tubular body; a wire; and a wire pipe, the first tubular body comprising an interstitial lumen between any present second tubular body; wire; and wire pipe, said interstitial lumen providing a fluid channel for flowing a fluid through the insertion cord, the insertion cord further comprising a distal tip provided by an elongated body part comprising a circumferential body surface and a distal end tip adjacent the circumferential body surface, an outer tubular sheath providing an external surface of at least a distal part of the insertion cord adjacent the distal end tip of the elon-gated body part, said outer tubular sheath being at least in part exposed to the interstitial lumen, wherein said outer tubular sheath provides at least one fluid opening for fluid to pass between the interstitial lumen and a surrounding of the insertion cord.

In an embodiment the at least one fluid opening is closed in a relaxed condition of the outer tubular sheath and opens when the outer tubular sheath is subject to a pressure of fluid introduced through the proximal end of the insertion cord. Hereby a valve function is obtained that prevents or limits back reflux of fluid into the interstitial lumen.

In an embodiment the outer tubular sheath is of a stretchable material adapted to be stretched when subject to a pressure of fluid introduced through the proximal end of the insertion cord. Hereby is obtained that stretching the material of the tubular sheath may allow the at least one opening to open and relaxation of said pressure may result in the stretchable material returning to its pre-stretched condition closing the at least one opening.

In an embodiment a distal portion of the outer tubular sheath is externally surrounding a part of the elongated body part and is preferably attached to the circumferential body surface thereof at separate positions circumferentially spaced around said circumferential body surface, thus leaving the outer tubular sheath unattached to the circumferential body surface at at least one position between spaced positions of attachment. Hereby may e.g. be obtained that irrigation fluid may leak out along the surface of the elongated body part in a direction towards the area in front of the distal end of the insertion cord. At the same time the attachments prevent displacement of the tubular sheath during insertion of the endoscope insertion cord.

In a further embodiment the outer tubular sheath, at least in a relaxed condition, surrounds the circumferential body surface with a tight fit. Hereby a valve function as mentioned above may be obtained.

In yet a further embodiment the elongated body part comprises at least one groove in the circumferential body surface, said at least one groove extending from a proximal end thereof to a distal end thereof, and said groove extending between positions of attachment of the outer tubular sheath to the circumferential body surface. Hereby the flow of irrigation fluid out of the interstitial lumen along the circumferential surface may e.g. be facilitated and focussed by an at least one channel provided by the at least one groove.

In an embodiment the at least one fluid opening is provided by a slit through the material of the outer tubular sheath. Such slit may provide an opening that is closed when the material of the outer tubular sheath is in a relaxed condition and that opens when subject to an internal pressure of fluid.

In an embodiment the distal part comprises a bending section. In such case the outer tubular sheath may be a cover of the bending section, and the material of the outer tubular sheath may be different from the material of the first tubular body.

In an embodiment the bending section extends distally beyond a distal end of the first tubular body, and in an embodiment the at least one opening is positioned distally of the distal end of the first tubular body.

In an embodiment the bending section comprises a row of segments, where adjacent segments are connected by hinges, preferably made integrally from the same material as the segments, the hinges being arranged for allowing the connected segments to bend in a bending plane, wherein the interstitial lumen extends through at least a number of the segments at a proximal end of the row of segments and the outer tubular sheath is exposed to the interstitial volume between adjacent segments. In a further embodiment the outer tubular sheath is attached to the most proximal segment through the entire circumference thereof to provide openings for the passage, e.g. exit, of fluid, e.g. irrigation liquid, only at one or more positions more distal than the position of the most proximal segment, possibly only at the distal end of the outer tubular sheath.

In a further embodiment the elongated body part comprises a distal segment of the row of segments.

In an embodiment the elongated body part includes a tip housing attached to the distal segment of the row of segments.

In an embodiment the outer tubular sheath is more flexible than the first tubular body. Thus the outer tubular sheath may be made of a different material than the first tubular body and/or the material of the outer tubular sheath may be thinner than the material of the first tubular body.

In some embodiments, the endoscope comprises a handle with an internal space, and an insertion cord, wherein the proximal end of the insertion cord is attached to the handle.

In some variations of the present embodiment, an interstitial lumen is provided in the insertion cord of the endoscope, the interstitial lumen flowing irrigation liquid to the distal end of the insertion cord.

In some variations of the present embodiment, the interstitial lumen of the first tubular body is sealed relative to the internal space of the housing.

In some variations of the present embodiment, said endoscope includes a seal between the interstitial lumen and an internal space of the handle. The seal may be positioned, at least partly, in the internal space. The seal may be as described with reference to embodiments according to the first aspect.

In some embodiments, an endoscope is provided that comprises a handle including a handle housing, an irrigation inlet port adapted to receive irrigation fluid, and an irrigation channel; and an insertion cord extending distally from the handle. The insertion cord includes an insertion tube; a bending section extending from the insertion tube; a distal tip extending from the bending section and including a tip housing; a camera at least partially enclosed in the tip housing; interstitial space within the insertion tube in fluid communication with the irrigation channel; and at least one interstitial flow opening in fluid communication with the interstitial space and adapted to discharge the irrigation fluid.

In a fifth aspect, the present disclosure provides a visualization system comprising an endoscope according to the fourth aspect, and a video processing device, such as a monitor.

In a sixth aspect, a method of irrigating a space within a patient is provided, the method comprising: inserting the insertion cord of the endoscope according to the first aspect into the patient; supplying irrigation fluid to the irrigation channel of the endoscope, the irrigation fluid flowing through the interstitial space and discharging through the at least one interstitial flow opening to irrigate the space.

### DESCRIPTION OF THE DRAWINGS

The disclosure will be explained in further detail by means of examples of embodiments having reference to the accompanying schematic drawings, in which:

FIG. 1a is a perspective view of an embodiment of an endoscope;

FIG. 1b is a perspective view of an embodiment of a video processing apparatus;

FIG. 1c is a front view of another embodiment of a video processing apparatus;

FIG. 1d is a perspective view of a portion of an insertion cord illustrating an interstitial space;

FIGS. 2a and 2b are schematic views of endoscopes including irrigation systems;

FIG. 3a is a perspective view of an embodiment of a handle of an endoscope;

FIG. 3b is a cross-section view of a portion of an embodiment of a handle showing an irrigation channel;

FIG. 4a is a perspective view of an embodiment of a bending section and distal tip;

FIG. 4b is a side view of the bending section of FIG. 4a;

FIGS. 4c and 4d are additional side views of the bending section of FIG. 4a;

FIGS. 5a to 5c are partial side and cross-section views of the bending section of FIG. 4a;

FIGS. 6a-6f are cross-section views of embodiments of bending sections;

FIGS. 7a and 7b are perspective views of an embodiments of a distal tip;

FIG. 8a is a cross-section view of the distal tip of FIGS. 7a and 7b;

FIG. 8b is a top view of a camera and light guides;

FIGS. 9a and 9b are views of an embodiment of a fluid junction body;

FIG. 10 is a view of another embodiment of a fluid junction body;

FIGS. 11 and 12 are perspective views of a further embodiment of a fluid junction body; and

FIG. 13 is an exploded view of an embodiment of an endoscope including components of the endoscope of FIG. 1.

### DETAILED DESCRIPTION

The present disclosure may be further understood with reference to the following description and appended drawings, wherein like elements are referred to with the same reference numerals.

Unless otherwise defined, all technical and scientific terms used herein have the same meaning as commonly understood by one of ordinary skill in the art. In case of conflict, the present document, including definitions, will control. Preferred methods and materials are illustrated below, although apparatuses, methods, and materials similar or equivalent to those illustrated herein may be used in practice or testing. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional terms that do not preclude the possibility of additional acts or structures. By contrast, the term “consists,” as used herein, is intended to be a closed-ended transitional term that precludes the possibility of additional acts or structures.

The term “distal,” as used herein, refers to a direction or position that is generally towards a target site, and the term “proximal,” as used herein, refers to a direction or position that is generally away from the target site.

It is noted that the “hollow outer cable housing” may also be denoted “wire pipe” or “wire tube”. Henceforward the term “wire pipe” will be used in this disclosure as a synonym to “hollow outer cable housing”.

As used herein the term “interstitial lumen” refers to the part of the inner lumen of first tubular body not occupied by any second tubular body; wires; or wire pipes, etc.

As used herein the term “snugly” refers to a situation wherein a surface of an element is close to a surface of another element either to be in direct contact or to leave a play between the two surfaces to provide for addition of a liquid or viscous pre-set sealing glue for said glue to provide for sealing in a set condition between the two surfaces. Especially in case of a cylindrical element, such as a tubular element, “snugly” refers to a situation where a surface of an element extends at least partially around the cylindrical element substantially following the contour of the surface of the cylindrical element.

As mentioned above, existing endoscopes and other visualization devices rely on dedicated channels, or lumens, for either irrigation or suction of fluid. Dedicated channel, as used herein, means a channel that is provided by structure that isolates the fluid flowing through the structure such that the fluid only contacts an internal surface of the structure, thereby the structure is dedicated, at least temporaly, exclusively to the passage of the irrigation fluid. For example, the channel may be defined by the internal surface of a tube or the internal surface of a tubular passage.

There is a need to minimize the outer diameter of the insertion cord to reduce trauma to the patient when the device is inserted, for example into the ureter. Ureteroscopes typically have outer diameters of approximately between 7-10 (e.g., 8-10) French (Fr). The smaller the outer diameter becomes, the less room there is to fit multiple dedicated working channels, e.g. one for irrigation and one for suction. Furthermore, as the tip end becomes smaller, it becomes more difficult to fit the camera, light source, and distal ends of various channels. One way to reduce the outer diameter is to eliminate a dedicated irrigation channel and use, instead, an interstitial lumen.

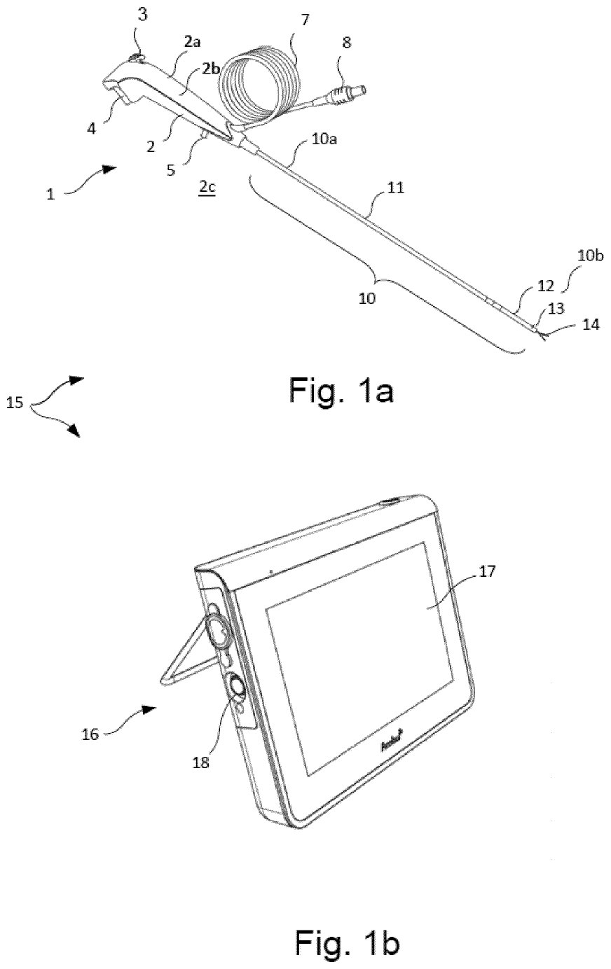

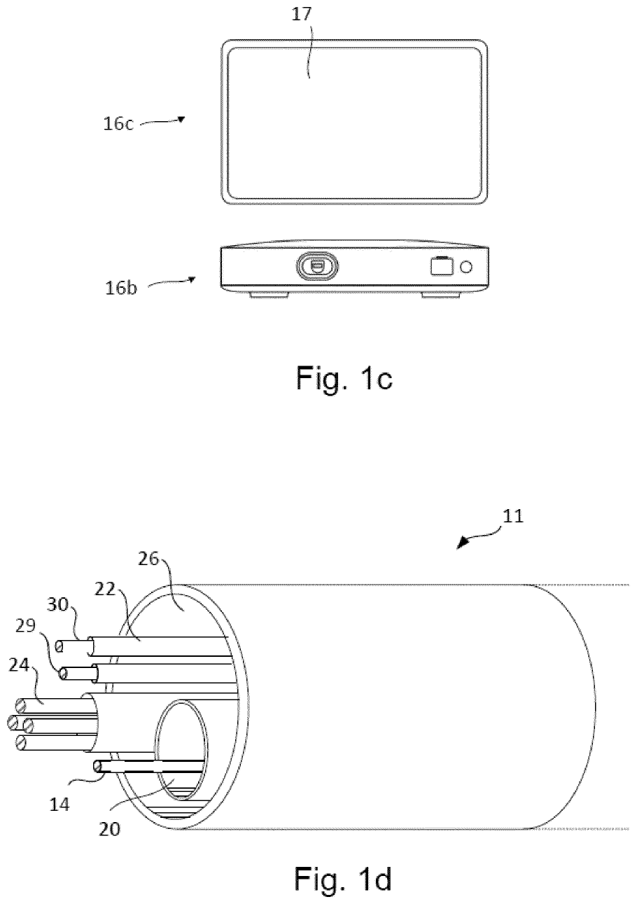

An interstitial channel or lumen, as used herein, means a channel formed in the internal space of a structure that also includes other components of the device, therefore the internal space is not dedicated to the passage of the irrigation liquid, which flows around, and contacts, the other components. See for example FIG. 1d.

While the present disclosure may be exemplified with an ureteroscope and methods of using the ureteroscope for treating kidney stones, the apparatus and methods described herein find use with any visualization device benefitting from a reduced cross-section or additional functionality, said visualization devices including, but not being limited to, endoscopes. Accordingly, references to the kidney and kidney stones can be generalized to tissue of the patient and objects, including kidney stones, debris and tissue.

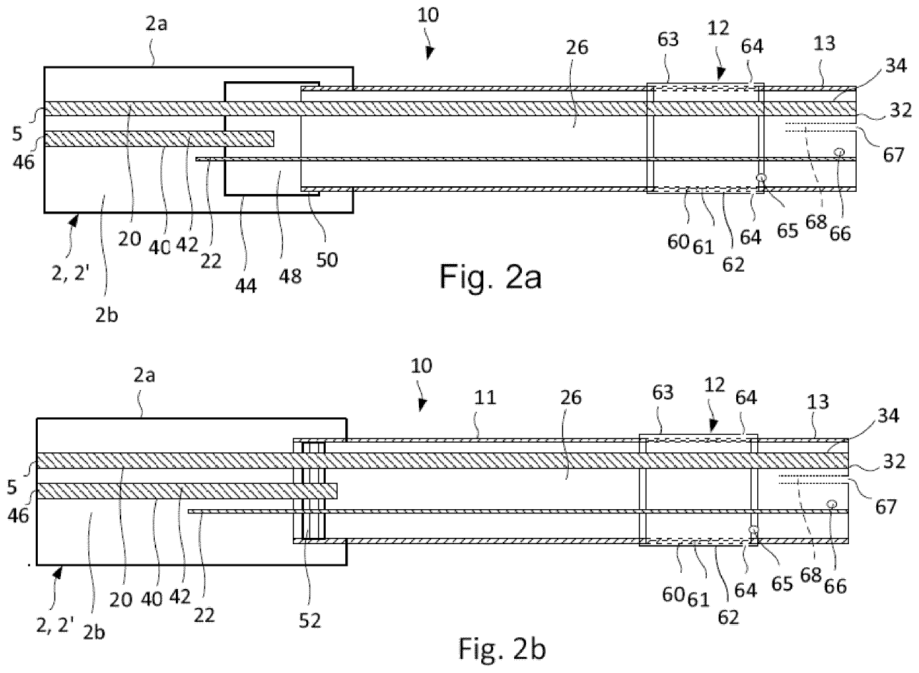

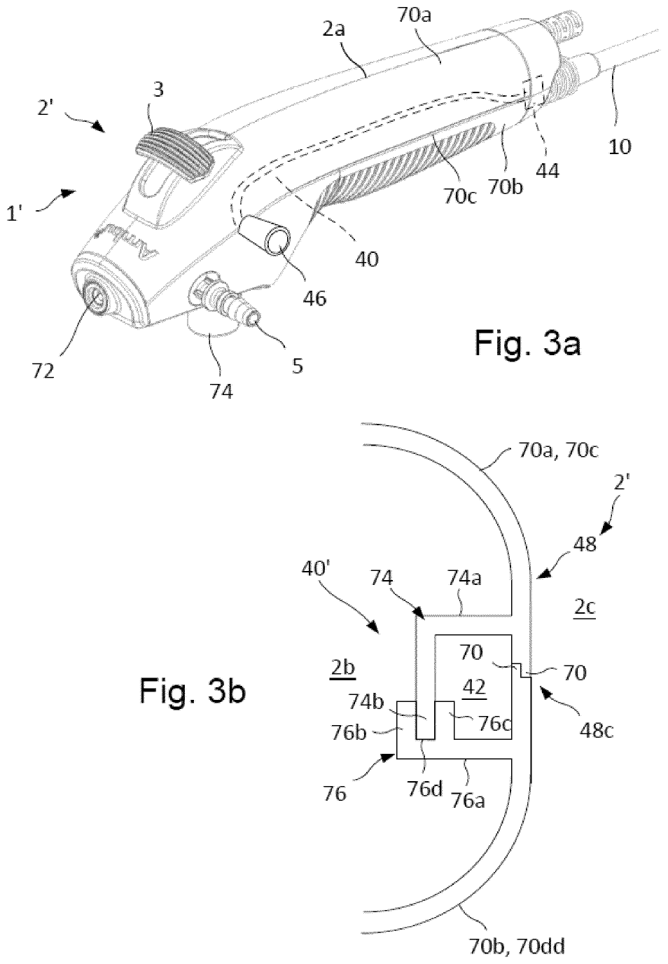

Provided herein in some embodiments is a device that utilizes the interstitial space, e.g. interstitial lumen, inside the insertion cord to deliver irrigation fluid at or near the distal end of the device while simultaneously using the working channel for fluid suction and/or other device components. This greatly improves visualization for the procedure while enabling a smaller device outer diameter. In practice, irrigation fluid can be pressurized and flow from an irrigation inlet port 46 in the handle, as described with reference to FIGS. 2a, 2b and 3a, through the interstitial space within the insertion cord, and exit the endoscope through the one or more interstitial flow openings. In some embodiments, these openings are at the distal tip to help clear away debris from the field of view, although the present disclosure is not limited to a particular location. Examples include, but are not limited to, on the top surface of the tip, the side surface of the tip, through an opening on the insertion tube or the bending section, or a combination thereof.

FIG. 1a shows a visualization device, illustratively an endoscope 1, having a position interface, illustratively a handle 2, and an insertion cord 10 attached to the handle 2. The insertion cord 10 includes an insertion tube 11, a bending section 12, and a distal tip 13 including a camera. An optional tool 14 is shown. The insertion cord has a proximal end 10a and a distal end 10b. The handle 2 comprises a handle housing 2a separating an interior space 2b of the handle 2 from an exterior space 2c. The handle 2 has a control lever 3 which enables an operator of the endoscope 1 to control the bending of the bending section 12 through pull wires, which by being tensioned or slacked can bend the bending section in one plane. The bending section could alternatively be controlled by pull wires in two planes. A suction button 4 and a suction port 5 are also shown. Upon actuation of the suction button 4 vacuum is applied to a working channel connected to the suction port. Fluids suctioned through the distal tip 13 are thereby drawn out and discharged via the suction port 5. A source of vacuum (not shown) is also connected to the handle 2. An image captured by the camera can be transmitted via a cable 7 having a cable connector 8 to a video processing apparatus 16 (shown in FIG. 1b). Example video processing apparatus include apparatus with and without integrated display screens. A video processing apparatus with an integrated display screen can be referred to as a monitor. A display screen can be connected to the video processing apparatus via a cable, e.g. HDMI or ethernet, or wirelessly. The visualisation device and the video processing apparatus can also include transceivers to communicate images and configuration data between them wirelessly, for example via a wireless HDMI protocol.

A position interface functions to control the position of the insertion cord. A handle is an example of a position interface and, unless stated otherwise, the terms are used interchangeably. The handle also functions to provide manual control actuators, e.g. knobs, levers, buttons, and the like, to steer the tip part and control instruments guided through the insertion cord. Alternatively, a different position interface can be provided that is connected to the insertion cord and is detachably connected to a robotic arm. The insertion cord thus extends from the robotic arm, and the visualization device is detachable from the robotic arm. The tip part (described below) is the same regardless of the position interface used. The robotic arm responds to signals, including voice commands from an operator, to rotate, translate, and otherwise position the proximal end of the insertion cord, as an operator would do manually. The position interface can include control actuators, including manual control actuators. Alternatively or additionally, control actuators can be provided in or on the robotic arm or by the robotic system including the robotic arm, thereby potentially reducing the cost of the visualization device. Example control actuators include single axis actuators, including linear motion actuators. A linear motion actuator may comprise a threaded rod coupled to a threaded nut portion, in which a motor rotates the rod to translate the nut portion.

A visualization system 15 comprises a visualization device and a video processing apparatus, for example the endoscope 1 and a monitor 16 or VPA 16a (shown in FIGS. 1b and 1c).

FIG. 1b shows a video processing apparatus, i.e. monitor 16, for displaying the image obtained by the camera of the endoscope 1. The monitor 16 includes a display screen 17 and a connector port 18. The cable connector 8 may be connected to the connector port 18 to present live images, obtained by the camera, with the display screen 17. Aspects of the endoscope 1, in particular aspects relating the bending system, control lever connection to the pull wires, and other aspects are known from commonly owned U.S. Patent Publication Nos. 2020/0138268 and 10,631,716, which are incorporated herein by reference.

Another embodiment of a video processing apparatus (VPA), i.e. VPA 16b, is shown in FIG. 1c, together with a display device 16c including the display screen 17 and the connector port 18 (not shown). The monitor 16 and the VPA 16b may perform the same video processing functions. The cable connector 8 may be connected to the connector port 18 to present live images, obtained by the camera, with the display screen 17. Aspects of the endoscope 1, in particular aspects relating the bending system, control lever connection to the pull wires, and other aspects are known from commonly owned U.S. Patent Publication No. 2020/0138268 and U.S. Pat. No. 10,631,716, which are incorporated herein by reference.

FIG. 1d is a schematic diagram illustrating a cross-section of an example insertion tube 11, such as the insertion tube of the visualisation device illustrated in FIG. 1a. The insertion tube 11 comprises a working channel 20 and pull cable sheaths 22. The working channel 20 is configured to receive the instrument 14. For example, the instrument 14 may be inserted through the working channel 20 and used to take samples of tissue, i.e. biopsies, near the distal tip. The working channel 20 can also be used to suction fluids through the distal tip.

One or more electrical conductors 24 provide power to the camera and light source at the distal tip and transfer data comprising, for example, camera configuration parameters and images obtained by the camera. The electrical conductors 24 may also be utilized to control the light sources at the tip part, such as to illuminate an area in front of the camera. In alternative examples, optical fibres are provided to substitute light sources at the distal tip and, to the extent used, respective power wires.

An interstitial lumen 26 is shown. The interstitial lumen 26 comprises the spacing within the inner surface of the insertion tube 11 that is not occupied by components of the insertion cord 10, for example the working channel 20, the pull cable sheaths 22, and the electrical conductors 24. The electrical conductors 24 comprise power wires and signal wires to provide communication capabilities via, for example, a serial communications protocol.

Pull or steering wires 29, 30 extend through the pull cable sheaths 22 in the insertion tube 11 from a bending actuator at the position interface, e.g. control lever 3, to the distal tip, whereby rotation of the bending actuator translates the steering wires 29, 30 causing the bending section to bend. Additional steering wires may be added for two dimensional steering. The pull wires 29, 30 may be solid wire, such as a monofilament steel wire, or a multifilament cable.

Having described, generally, the visualization device and VPA, attention is now directed to FIGS. 2a-3b, which are are schematic and perspective views of embodiments of endoscopes including irrigation systems as described in the preceding paragraphs and the figures below. Generally, the endoscopes include the handle 2, 2′ (handle 2′ shown in FIG. 3a), the insertion cord 10 including the insertion tube 11, the bending section 12, and the distal tip 13. A working channel port 32 is provided at the end of the working channel 20. A working channel tube 34 may define part of the working channel. The working channel port 32 is typically molded in a tip housing of the distal tip. The working channel port 32 can also be provided, if the working channel tube 34 extents to the distal end of the distal tip, by the working channel tube 34 without a molded portion. The working channel port can be considered to be the portion of the tube or molded portion that defines the opening. In the figures below the working channel port is discussed in the context of its position in various planes and in relation to other components at the distal tip 13.

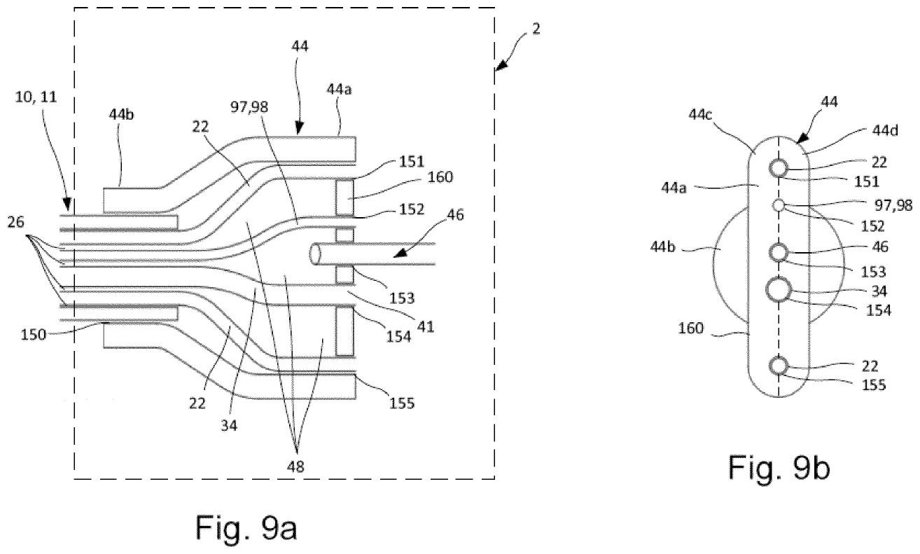

An irrigation tube 40 defining within it a portion of an irrigation channel 42 is provided in the handle 2, 2′. The irrigation tube 40 extends to a fluid junction body 44 (described with reference to FIGS. 9a-12b) establishing fluid communication between an irrigation inlet port 46 (see FIG. 3a), an internal space 48 of the fluid junction body 44, and the interstitial space 26 of the insertion tube 11. The fluid junction body 44 may receive the proximal end of the insertion tube 11 and be fluid sealed thereto at a seal 50. In FIG. 2b the fluid junction body comprises a seal body 52 positioned at the proximal end of the insertion tube 11 and the irrigation tube 40 extends through the seal body 52 into the insertion tube 11. In both embodiments the irrigation channel 42 is comprised by the irrigation tube 40 (or integral irrigation tube 40′ shown in FIG. 2a) and the interstitial space 26.

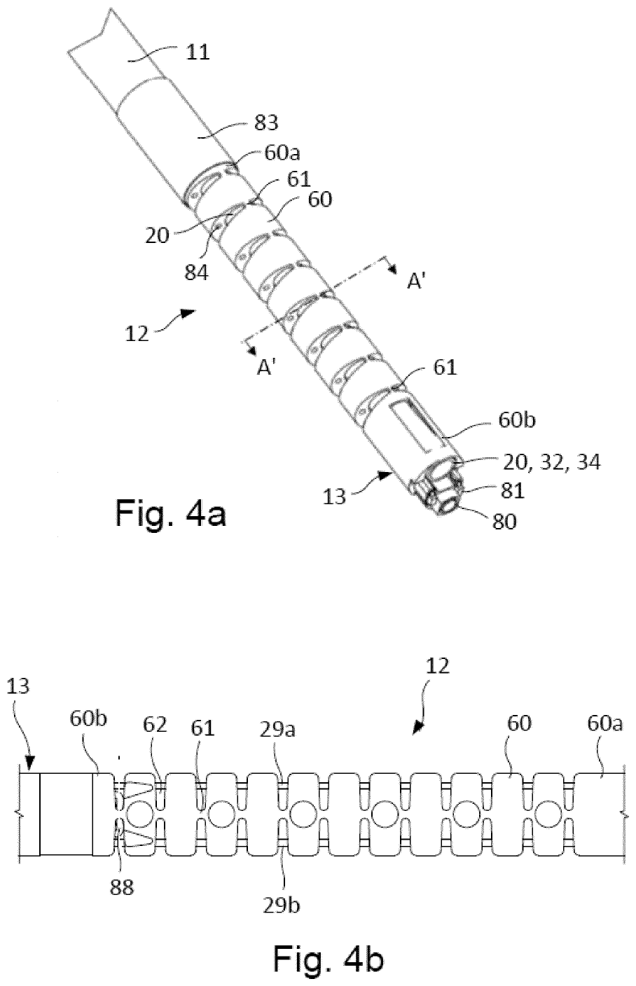

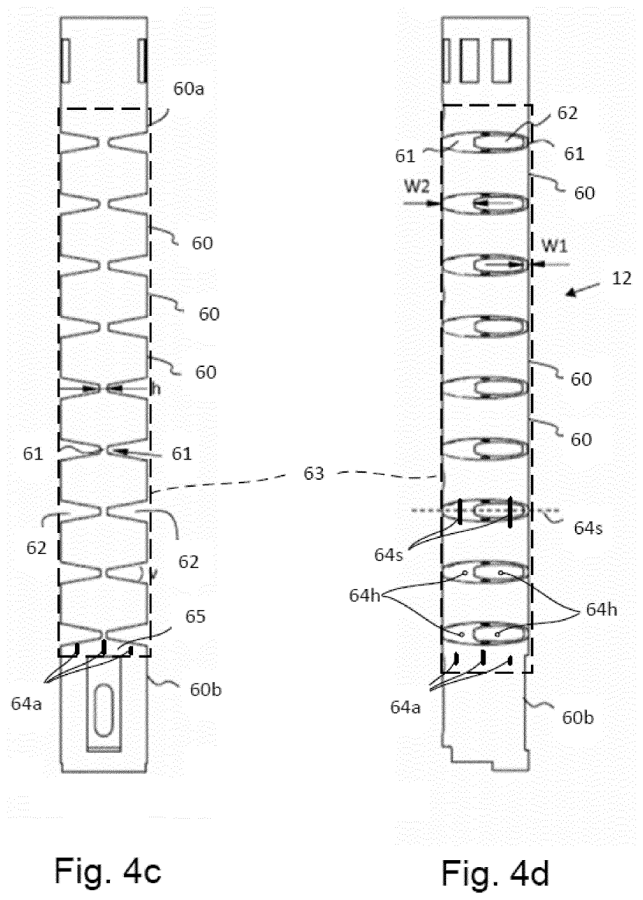

FIGS. 2a and 2b also show features of the bending section 12, which includes segments 60 connected by hinges 61 and defining open spaces 62 between adjacent segments 60. A bending section sleeve 63 extends over the bending section 12 and is bonded at both ends to the bending section 12 to enclose and fluid seal, except with respect to interstitial flow openings, the open spaces 62. Interstitial flow openings 64, 65, 66, and 67 are shown. The bending section 12 is also shown, in additional detail, in FIGS. 4a to 4d, FIG. 4a being a perspective view, and FIGS. 4c and 4d showing the bending section 12 rotated 90 degrees to best show the hinges 61 and the interstitial flow openings 64. The bending section sleeve 63 is shown in dashed lines, representing transparency, so the segments of the bending section are visible therethrough in the figures.

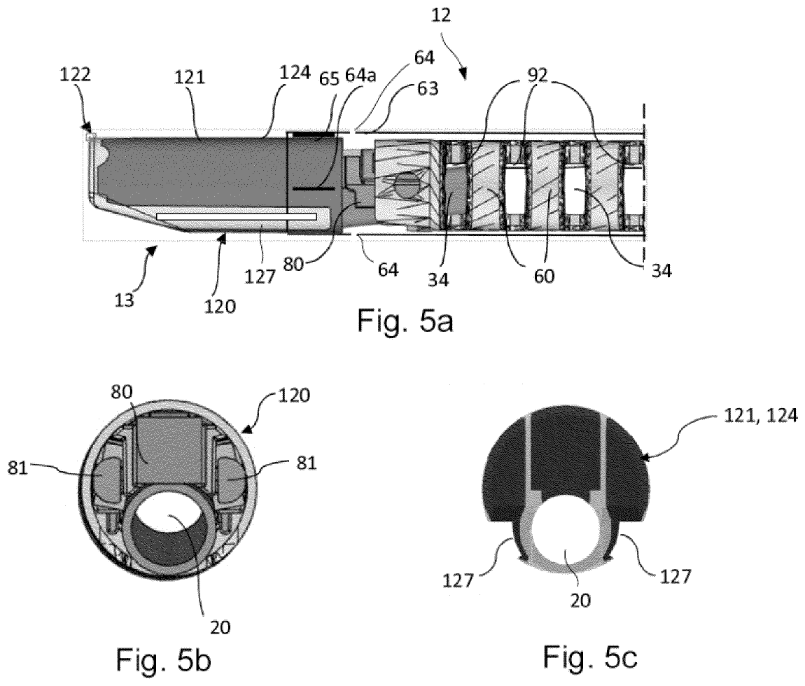

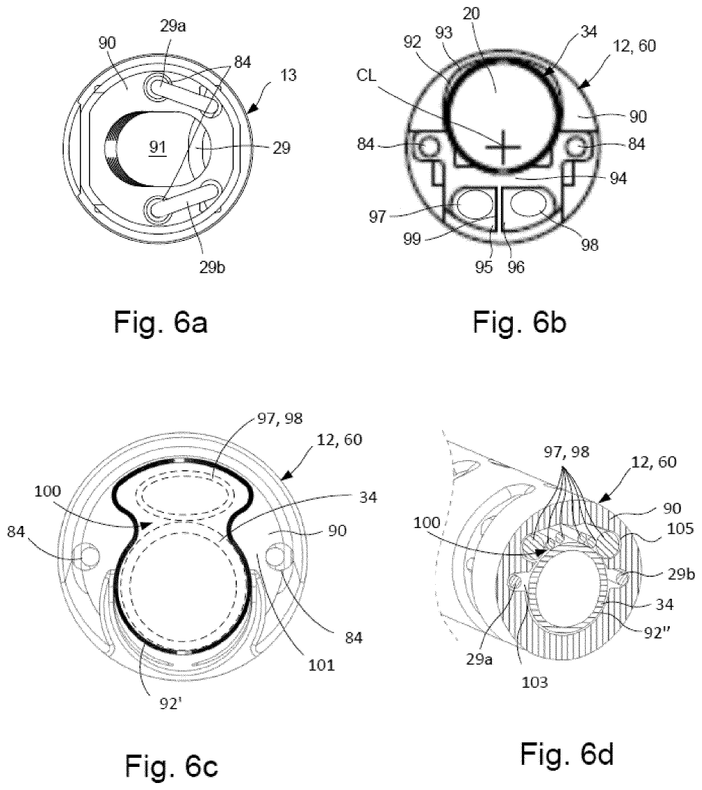

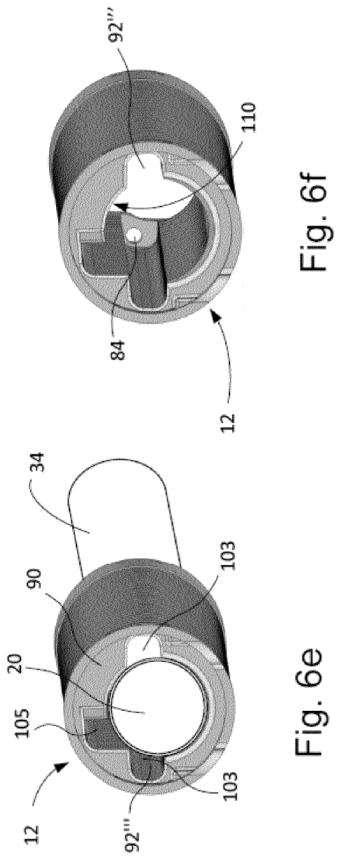

The interstitial flow openings 64 are formed in the bending section sleeve 63 and may comprise holes 64h, slits 64s, or any other shapes, as depicted in FIG. 5e. The interstitial flow opening 65 is formed between the bending section sleeve 63 and a segment 60 or the tip housing, preferably the distal segment. The interstitial flow opening 66 is formed in a wall of the distal tip 13. The interstitial flow opening 65 is formed in the distal segment or the distal tip. The interstitial flow opening 67 is formed in a front or distal wall or window of the distal tip 13. A portion of the irrigation channel 42 is provided by the interstitial space 26. Another portion of the irrigation channel 42 can be provided by forming or molding an elongate cavity, 68, in the distal tip 13, which may be referred to as a distal tip irrigation channel portion 68. The irrigation channel 42 and flow openings are further disclosed with reference to FIGS. 5a, 6a-d, 7b, and 8a.

FIG. 3a shows another embodiment of a handle of an endoscope, denoted by numerals 1′ and 2′ respectively. The handle 2′ is in most respects the same as the handle 2, except as described herein and below. The handle 2′ comprises the irrigation inlet port 46 connected via the irrigation tube 40 to the fluid junction body 44 (described with reference to FIGS. 9a-12) fluidly connecting the irrigation inlet port 46 to the interstitial space 26 of the insertion tube 11. The handle housing 2a comprises mating housing parts 70a and 70b which form a housing joint 70c. Also shown are a working channel inlet port 72, a fluid discharge port 74, and the suction port 5. Tools can be inserted through the working channel inlet port 72. When suction is applied via a suction channel, the fluid is discharged via the fluid discharge port. The suction port 5 is connected to a vacuum source that can be engaged to provide suction via working channel port port 32. The structure connecting the working channel inlet port 72 and the suction port 5 with the working channel 20 is conventional and will not be described in additional detail.

The irrigation tube 40 may, in an alternative embodiment shown in FIG. 3b and denoted with numeral 40′, be integrally molded with the housing 2a, which comprises internal walls 74, 76 sized and shaped to form the integral irrigation tube 40′. The irrigation channel 42 may extend from the irrigation inlet port 46 to the fluid junction body 44. The mating housing parts 70a, 70b include peripheral walls 70c, 70d, respectively, and internal walls 74, 76 extending therefrom toward the internal space 2b. The internal walls 74, 76 connect with each other defining the integral irrigation tube 40′.

The internal walls 74, 76 may connect with each other by interlocking to ensure a fluid seal for the irrigation channel 42. In some variations, the interlocking may be provided by two wall portions of one of the housing parts forming a channel for a wall portion of the other of the housing parts. An example of such an interlocking is provided by wall portions 76b and 76c of the internal wall 76 providing a channel or groove 76d for a wall portion 74b, or tongue, of the internal wall 74, which extends from a wall portion 74a connecting the peripheral wall 70c with the tongue 74b. The channel interlocking, known as a “tongue and groove” joint, may also be formed on the peripheral walls 70c, 70d. A wall portion 76a is also shown, from which the wall portions 76b and 76c extend to form the channel 76d.

In some variations, the interlocking may be provided by a wall portion of one of the housing parts mating, without forming a channel, with a wall portion of the other of the housing parts. An example of such a channelless interlocking is provided by wall portions 70e and 70f of the peripheral walls 70c, 70d, where each of the wall portions 70e and 70f overlaps the other. Each of the wall portions 70e, 70f abuts the opposite peripheral wall portion 70e, 70f. Their thickensses in the overlapping portion may be less than the thickness of the peripheral wall, preferably both thicknesses adding up to the thickness of the peripheral wall. The channelless interlocking, also known as a “half lap” joint, may also be formed on the internal walls 74, 76 instead of the tongue and groove joint.

As shown, the tongue and groove joint provides a more secure seal, protecting the internal components of the handle 2, 2′ from irrigation fluid leaks while the half lap joint keeps the irrigation fluid from leaking to the outside space. However, the stresses provide by the tongue and groove joint on the internal walls can also press the portions of the half lap joint against each other to improve the external seal. Of course an adhesive may be applied along the half lap and/or tongue and groove joints to further seal the irrigation channel 42′.

Having discussed the handle attention is now directed to the bending section and the distal tip of the insertion cord 10. FIG. 4a shows an embodiment of the bending section 12 and the distal tip 13. Here, the bending section sleeve or cover 63 is not shown to clearly show the structure of the bending section. Also, the molding plastic housing of the distal tip is not shown. The bending section 12 includes a number of segments including a distal end segment 60b, a proximal end segment 60a connected to the insertion tube 11, and a number of segments 60 arranged between the distal end segment 60b and the proximal end segment 60a. The segments 60, 60a, 60b are interconnected by hinges 61. These hinges 61 are preferably integrally made from the same material as the segments, and preferably the hinges and the segments form a single continuous piece of material. Examples of materials could be polypropylene, polyacetal (POM) or a semi-aromatic polyamide (nylon). The distal tip 13 comprises a camera 80, light emitting diodes 81 and the working channel port 32. A sleeve 83 may be used to secure the bending section 12 to the insertion tube 11, for example with an adhesive or press-fit or both. The sleeve 83 may be part of the proximal end segment 60a. The protective cover or sleeve 63 of the bending section 12 can abut the distal edge of the sleeve 83 and be bonded to the proximal end segment 60a to fluidly seal the proximal end of the bending section. As shown, the distal tip 13 comprises the distal end segment 60b. The distal tip 13 can also be connected to but not comprise the distal end segment 60b. The bending section 12 can be used with the embodiments of endoscopes described with reference to FIGS. 1a, 2a, 2b, and 3a and also with the endoscopes described below. FIG. 4a also shows passages 84 for positioning of the pull wires 29, 30. The pull wire sheaths 22 may terminate at the proximal end segment 60a, and the pull wires continue through the passages 84 in the bending section, which form a guiding channel for the pull wires and are preferably placed close to the outer periphery of the bending section. The pull wires extend from the handle 2, 2′ to the distal end segment 60b of the bending section. The pull wires are secured to the distal end segment 60b or to the distal tip 13. This configuration together with the hinges 61 allows for the bending of the bending section by tensioning one pull wire and slacking the other.

FIG. 4b illustrates a pull wire 88 having proximal sections 29 and 30. In this example a single wire is attached to the distal end segment 60b and the proximal sections of the wire function as described with reference to pull wires 29, 30. The wire can also be attached or affixed to the distal tip 13. Of course, individual wires 29, 30 secured to the distal end segment 60b or to the distal tip 13, for example by potting, can also be used. The open spaces 62 between segments 60 are also shown.

FIGS. 4c and 4d show additional bending section details. In FIG. 4c the plane with hinges 61 is perpendicular to the plane of the paper. A number of wedge formed open spaces 62 are formed between the segments 60 to allow for bending of the bending section. When the bending section is in the neutral (straight) position, the angle of the open spaces 62 is v as indicated. When the bending section is bending the angle v will be smaller on one side and larger on the opposite side. The letter h represents the height of one of the hinges. The hinges can have different heights along the length of the bending section. The width is indicated by letters W1 and W2, which represent hinges on one side being at least twise as wide as the hinges on the opposide side. Width W1 corresponds the wall thickness of the external wall forming the segments. Width W2 corresponds to a hinge that extends into the interior space of the bending section from an internal surface fo the external wall. Preferrably, the slits 64s are longitudinally aligned to irrigate. In FIG. 4d the bending section 12 is rotated 90° around the longitudinal axis. Additional details pertaining to the bending section and the endoscope, except for features pertaining to the interstitial spaces and related structures and functions as claimed herein, are disclosed in International Application Publication No. WO 2019/002186 A1, which is incorporated herein by reference.

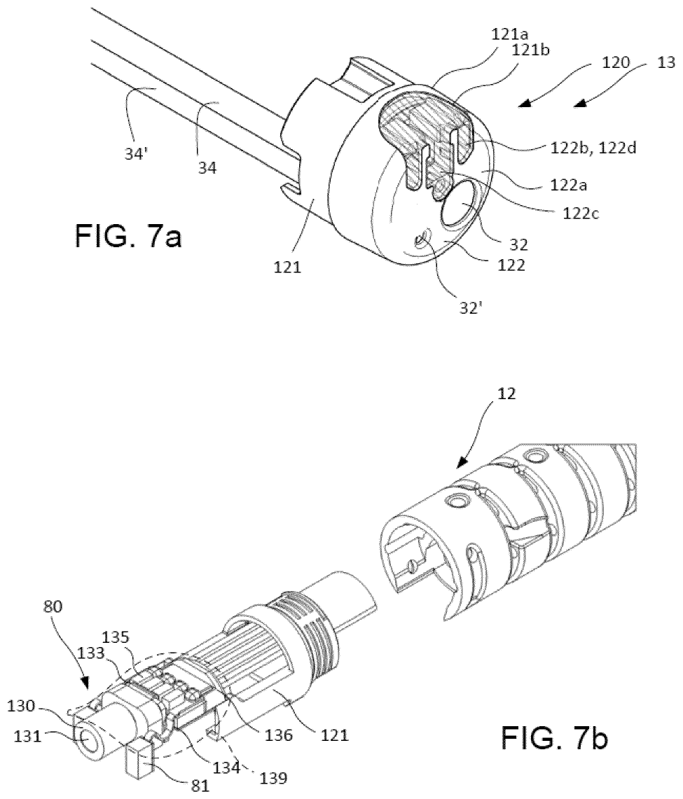

FIGS. 5a-c illustrate embodiments of the interstitial fluid openings. Shown are the bending section 12 and the bending section sleeve 63. The distal tip 13 comprises an elongate tip housing 120 connected to the distal segment 60b and having a circumferential wall 121 and a distal, or front, wall 122 connected to the wall 121. The tip housing 120 is also described with reference to FIG. 7a. The wall 121 has an outer surface 124. As shown, the distal segment 60b is not yet connected to the tip housing 120, for illustrative purposes. The bending section 12 has interstitial space also, provided by space 92, 100, 103, 105 (best seen in FIGS. 6b-6d) between the various apertures, tubes, and components passing therethrough. The interstitial space in the bending section is in fluid communication with the interstitial space 26 of the insertion tube since the insertion tube 11 and the proximal segment 60a are connected at their peripheries. Each of the embodiments described herein describing interstitial fluid openings may be combined with the other embodiments to define a desirable irrigation system.

Referring to FIG. 5a, in one embodiment, the interstitial fluid openings 64 are formed by slits in the bending section sleeve 63. The interstitial fluid openings 64 may open when fluid pressure is applied. The slits can be oriented in various orientations. An axial or longitudinal orientation is preferred.

Still referring to FIG. 5a, in another embodiment, the bending section sleeve 63 is bonded to the outer surface 64 at discrete positions 64a and not bonded between the bonding positions 64a, which permits pressurized irrigation fluid to discharge through the interstitial fluid openings 65 formed by the unbonded portions of the bending section sleeve 63 overlapping the outer surface 64. The number and sizes of the bonding sites determine the size of the interstitial fluid openings 65 and the pressure required to irrigate. The bonding sites are circumferentially spaced around the circumferential surface, e.g. by gluing or welding. All other disclosed types of interstitial fluid openings can also be included. At the opposite end, the bending section sleeve 63 is bonded to the bending section to seal the connection between the proximal end of the bending section sleeve 63 and the bending section 12.

FIGS. 5b and 5c are cross-sections of the distal tip 13 showing the camera 80 and the LEDs 81 inside the peripheral wall 121 of the tip housing 120. The working channel 41 is also shown. Referring to FIG. 5c, the tip housing 120 may comprise, formed on its circumferential wall 121 and the outer surface 124, one or more grooves 127 extending from a proximal end thereof to a distal end thereof, the grooves extending between bonding positions 65. The grooves 127 may be positioned at places on the tip housing 120 where internal components, such as the working channel, cables, or wires, are not present directly adjacent to the internal surface of the circumferential wall 121 thus allowing space for such grooves. These grooves 127 will act as external irrigation channels for fluid discharged through the respective interstitial fluid openings 65. The cross-sectional shape, and longitudinal shape of the grooves 127 will control the irrigation fluid as it discharges and can be designed to form narrow or wide plumes of fluid in addition to directing an orientation of the plumes/discharged fluid. The grooves 127 may extent in the axial direction or follow a curved or skew path as space allows depending on the position of subjacent components.

The interstitial fluid openings of the preceding embodiments may be closed in a relaxed condition of the bending section sleeve and open when the bending section sleeve is subject to a pressure of irrigation fluid introduced through the proximal end of the insertion cord 10 through the interstitial space. The bending section sleeve may comprise a stretchable/resilient material adapted to be stretched by the fluid pressure to open the interstitial fluid openings, which may be referred to as pressure-dependent interstitial fluid openings. The length of the slits and the thickness and tensile strength of the material can be selected for a desired irrigation pressure.

The interstitial fluid openings, especially when the openings are provided at positions of non-attachment, as described above, and the outer tubular cover in a relaxed condition surrounds the circumferential surface of the housing 120 with a tight fit, the interstitial fluid openings may close when the the internal or irrigation pressure is below a threshold, which may be a no-pressure threshold. Further, when the interstitial fluid openings are provided by slits, and especially when such slits extend in a longitudinal direction of the insertion cord, the slits 43 may close in the relaxed condition of the outer tubular cover and open when subject to an internal pressure of irrigation liquid in the interstitial space of the bending section.

The cross-section of the distal tip can have several configurations, some of which are more advantageous for creating larger interstitial space in the distal tip. Referring to FIG. 6a, in one embodiment the distal end segment 60b or the distal tip 13 comprise a wall 90 with a central opening 91. The pull wire 29 is bent in the manner shown in FIG. 6a, through and from one side to the other and back, of the wall 90 through passages 84, to provide a simple way to secure the pull wire 29 to the distal end segment 60b or the distal tip 13. The working channel tube can pass through the central opening 91. The proximal sections 29a, 29b of the pull wire 29 are also shown. The spacing between the proximal sections 29a, 29b and the passages 84 and between the working channel tube and the central opening 91 provide interstitial space to the distal tip and the bending section.

FIG. 6b is a cross-sectional view (A-A in FIG. 4a) of an embodiment of the bending section. The longitudinal center axis is indicated by a cross CL. The passage 92 for the working channel tube 34 is also shown. This will often have a circular shape. Passages 84 for the pull wires are also seen. These are often arranged in opposite directions from the longitudinal center axis CL and will extend perpendicularly to a plane traversing the tube 34, referred to as the bending plane when the endoscope bends in one dimension. Hinges 61 are positioned adjacent the bending plane. Two further passages 95 and 96 and exemplary components 97 and 98 are illustrated. These components represent, for example, electrical wires, such as signal cables, or additional tubing for an additional working channel. These two passages are separated by a hinge 99, referred to as a second, or internal, hinge, by contrast with a circumferential, or external, hinge 61.

In the present embodiment, the passage 92 in the wall 90 accommodates the tube 34. Some space 93 may remain between the circumference of the passage 92 and the tube 34. The space 93 may increase the flexibility of the bending section and also add volume to the interstitial space. The interstitial volume also may include the space between the components 97, 98 and the passages 95, 96 and between the pull wires 29a, 29b and the pull cable passages 84. A wall portion 94 separates the tube 34 and the passages 95, 96.

FIG. 6c is a cross-sectional view of another embodiment of the bending section. By contrast with the embodiment described with reference to FIG. 6b, in the present embodiment the wall portion 94 has been removed and the components 97, 98 and the tube 34 pass through a larger passage 92′. Between the components 97, 98 and the tube 34 is additional interstitial space 100. A wall portion 101 separates the pull wire passages 84 from the larger passage 92′.

FIG. 6d is another cross-sectional view of another embodiment of the bending section, which is similar to the embodiment described with reference to FIG. 6c in that the wall portion 94 has been removed and the components 97, 98 and the tube 34 pass through a larger passage 92″. Additionally, the wall portion 101 has been removed and therefore the pull wires 29a, 29b also pass through a common passage 92″. The removal of wall passages increases the interstitial space. Cut-outs 103 and 105 are also shown and are described below.

FIGS. 6e and 6f are cross-sectional views of another embodiment of the bending section. In the present embodiment the passage 92″ comprises a substantially circular portion 110 for the tube 34 and two or more cut-outs in the wall 90, illustratively cut-outs 103 and 105, which provide channels through which the components 97, 98 and the pull wires 29a, 29b pass. The bending section thus provides, in a manner similar to the embodiment described with reference to FIG. 6d, structure for the internal components without wall portions that reduce the interstitial space.

FIGS. 7a and 7b are perspective views of an embodiment of the distal tip 13. In the present embodiment the distal tip 13 includes the tip housing 120 to be connected to the distal segment 60b and having the circumferential wall 121 and the front wall 122 connected to the wall 121. In some variations the circumferential wall 121 comprises a transparent material. As shown, the circumferential wall 121 has a first portion 121a made of a first material and a second portion 121b made of a second, transparent, material. Portions 121a and 121b can be made in one piece in a two-shot injection molding process. Portions 121a and 121b can also be made in two pieces and bonded together to form a single piece. The front wall 122 has a first portion 122a made of the first material and a second portion 122b made of the second, transparent, material. Portions 122a and 122b can be made in one piece in a two-shot injection molding process. Portions 122a and 122b can also be made in two pieces and bonded together to form a single piece. Portions 121a and 122a are made in one piece and portions 121b and 122b are made in one piece, and all of them can be molded as one piece in the two-shot molding process. The second portion 122b includes a camera window 122c and a light window 122d. As discussed below, the light window 122d can be integrated with a light guide positioned between the LED and the light window, and the combination can be referred to as a light source. Of course light fibers can be used instead of the LED, in which case a light source is used to illuminate the light fibers, as is known in the art. Working channel ports 32 and 32′ extending from working channel tubes 34 and 34′ are also shown.

FIG. 7b presents a section of the circumferential wall 121 with a portion cut out for illustrative purposes. Within the tip housing 120 is the camera 80 and the LED 81. The camera 80 comprises a camera barrel 130 holding one or more lenses 131 therein and an image sensor 133 axially aligned with the camera barrel and mounted on a circuit board 134 which also may be, optionally, connected to a number of electronic circuits 135. A support frame 136 is provided to facilitate inserting the camera from the proximal end of the tip housing 120 with cables protruding proximally. The camera components may be potted, as illustrated by the dashed line 139, to keep them dry. The interstitial space may extend between the potting material, e.g. epoxy and curable polyolefin polymers, and the circumferential wall 121. Additionally or alternatively, the distal tip irrigation channel portion 68 may be formed in the epoxy to establish fluid communication between the interstitial space and the port 67, for example by potting a wire and then removing it.

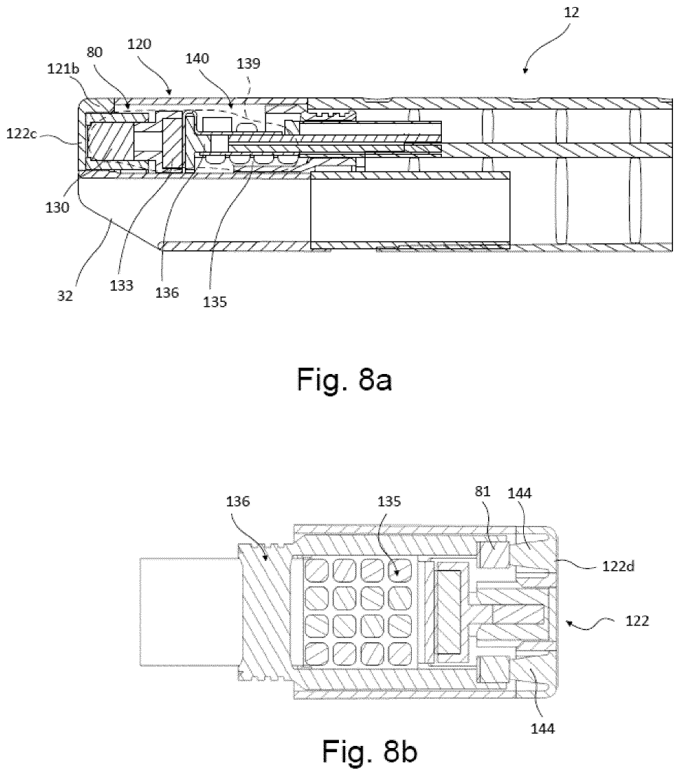

FIGS. 8a and 8b a views of another embodiment of the distal tip 13 which is similar to the embodiment of FIGS. 7a and 7b except for the angled working channel port 32. In the present embodiment the interstitial space created by potting is denoted by numeral 140. Light guides 144 are disposed between the LED 81 and the transparent portion of the front wall 122. In some variations potting does not extend to the light guides 144 to ensure air surrounds them, which improves internal reflection. In some variations cladding is applied to the light guides 144 to improve internal reflection and potting may extend to the cladding, but it does not have too. The light guides 144 can be formed with the transparent portion of the front wall, in one piece, or can be bonded, e.g. adhesively, to a flat front wall. Although not described in detail hereinabove or below, any of the described distal tip embodiments can include light guides as in the present embodiment and variations thereof described herein.

FIG. 9a is a partial longitudinal cross-section of an embodiment of the fluid junction body 44 and FIG. 9b is a proximal view of the same. The fluid junction body 44 comprises a proximal side 44a opposite a distal side 44b. The distal side 44b is sized and shaped to receive the insertion tube 11 and to be fluid sealed therewith. The proximal side 44a is proximal of the distal side 44b and is sized and shaped to receive the irrigation tube 46 and all the components extending from the handle 2, 2′ through the insertion tube 11 (referred to as the insertion tube components), and to be fluid sealed therewith. As shown, the insertion tube components include the pull cable sheaths 22, the working channel tube 34, and the components 97, 98. The irrigation tube 46 discharges irrigation fluid into the internal space 48 of the fluid junction body 44 and the irrigation fluid then flows, due to fluid pressure, into the interstitial space 26 within the insertion cord 10. Although shown in linear alignment, the proximal side 44a of the fluid junction body 44 does not have to be linearly aligned with the distal side 44b. Furthermore, the insertion tube components do not need to be parallel to each other and can be received by the fluid junction body 44 from any direction.

FIG. 9a also shows a number of passages 150-155, referred to as the junction body passages, for the insertion tube 11, the irrigation tube 34 and all the components extending from the handle through the insertion tube 11. The junction body passages can be sized to form seals with the respective components received by each passage. For example, the passages can be shaped to receive the component in a press-fit manner. An adhesive or other sealant may be applied to the passage so that after the component/tube is inserted the adhesive/sealant seals the passage. O-rings or tubes of material softer than the irrigation body may be interposed between the passage and the component to form a seal. The passages 151-155 are formed in a wall 160 of the fluid junction body 44, which may comprise two sides formed with each of sides 44c and 44d. Alternatively, the wall 160 can be a plug or grommet that is made separately and inserted into a cavity of the fluid junction body 44.

Referring now to FIG. 9b, in some embodiments the fluid junction body 44 is formed from two parts, 44c and 44d, assembled together. Each part includes an arcuate portion with a surface that is part of the surface of each of the junction body passages. When the two parts are bonded together, the junction body passages are formed by the union of the arcuate surfaces at a seam denoted by a dashed line. The two parts can be bonded in any known manner, including adhesive and ultrasonic bonding. In alternative embodiments, the fluid junction body 44 is formed in one body and the junction body passages are formed, for example by drilling with a drill or laser. The passages 111-115 may be formed in the wall 160 of the fluid junction body 44, which may comprise two sides formed with each of sides 44c and 44d. A sealant or adhesive can be applied to passages 151-155 to improve or form the seal.

In some embodiments, the fluid junction body 44 is constituted by a single molded body that may be molded in situ around the insertion tube 11, the pull cable sheaths 22, the working channel tube 34, and the components 97, 98 to seal them with the fluid junction body 44. Alternatively, the two parts 44c and 44d can be molded in a two-shot injection molding step after placing the pull cable sheaths 22, the working channel tube 34, and the components 97, 98.

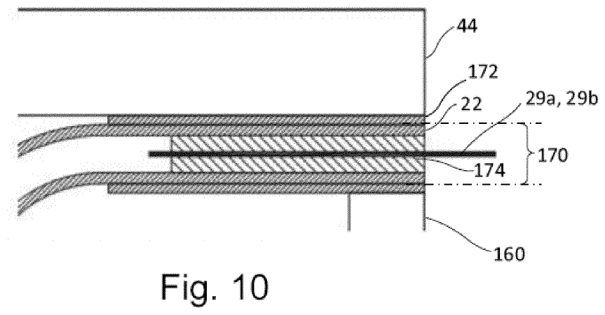

FIG. 10 is a partial cross-sectional view of an embodiment of a pull cable 170. Typically the pull cable 170 comprises a pull wire 29a, 29b inside a sheath 22. The sheath can be made of a coil and may include a polymeric coating thereon. As shown, a pipe sealant 172 is provided along a length and on the outer surface of the sheath 22. Another sealant, 174, is applied between the pull wire 29a, 29b and the inner surface of the sheath 22. The sealants may be applied along the sheath for a distance longer than the width of the wall 160 so that sealant extends from one or both sides of the wall 160 proximally, distally, or both proximally and distally. The sealant 174 may be a sealing glue. Slip means, such as oil, may be present between the sealing glue and the pull wire 29a, 29b. The sealant 172 may comprise an adhesive or tube of material softer than fluid junction body 44. Hereby, it is on one hand obtained that irrigation liquid does not leak through the wire pipe when this is provided by a wire pipe made of a wound wire whereby the wire pipe is not water tight and due to the slip means it is, on the other hand, possible for the pull wire to be pulled back and forth in spite of the presence of the sealing glue. In some examples, the pull cable 170 sealing is achieved by the sealing glue 174 penetrating the sheath from the inside thereof, particularly when the sheath is comprised of coiled wire. The sealing glue 174 thus fills spaces between portions of the coil. Of course the pull cable 170 extends proximally from the fluid junction body 44 and the sealant 172 may also extend proximally from the fluid junction body 44.

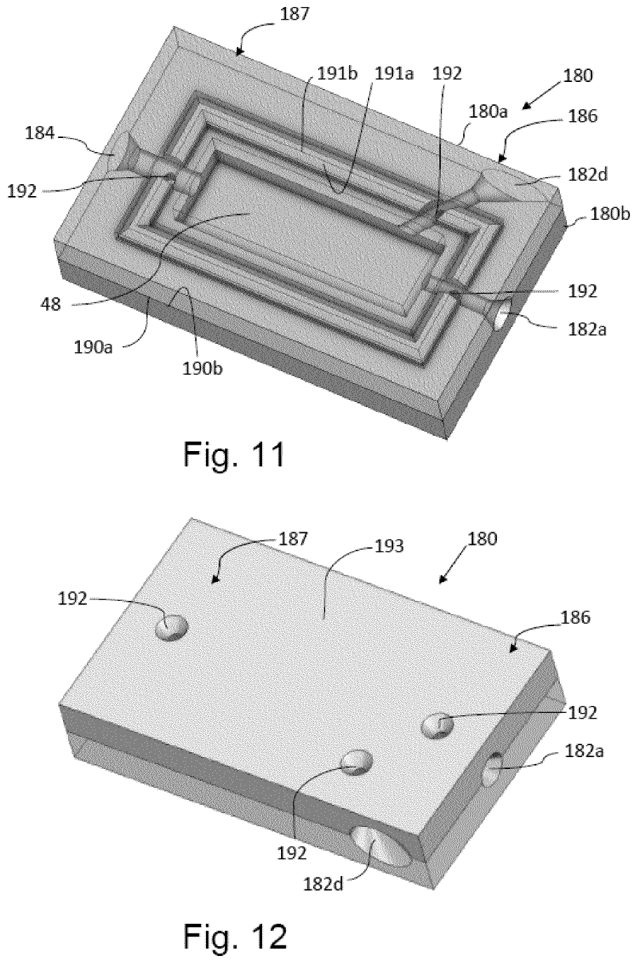

FIGS. 11 and 12 show another embodiment of a fluid joint body, denoted by numeral 180, comprising a first body part 180a and a second body part 180b. The first and the second body parts 180a, 180b are joined to form, for example, first, second, and third passages or channels 182a, 182d, and 184 provided for insertion of the insertion tube 11, the irrigation tube 46, and a tube 34 providing a working channel. More channels can be provided for pull cables and additional components. For sake of illustration the first body part 180a is shown as being transparent and the second body part 180b is shown as being opaque. The first and the second body parts 180a, 180b each comprises an assembly face 190a, 190b for connection with other of the second and the first body part 180b, 180a. The assembly faces 190a, 190b are respectively provided with mutually corresponding valleys 191a and ridges 191b for securing mutual positioning in the assembled condition when the assembly faces 190a, 190b are placed in mutual abutment, possibly with a layer of glue in between.

The first and the second body parts 180a, 180b each comprises a first portion and a second portion and in the assembled condition shown in FIGS. 11 and 12 the joint first portions and second portions together provide a first sealing body portion 186 and second sealing body portion 187. The first sealing body portion 186 comprises first parts of the first portions of the first and second body part 180a, 180b and said first parts provide together in the assembled condition of the sealing body, a first channel 182a for the at least one of a working channel tube 34, a pull cable 170 or a component 97, 98 to extend into the fluid joint body 180. Thusly, the first parts are intended for extending along at least a part of a circumference of at least one of any pull cable, tube 34, or component 97, 98.

The second sealing body portion 187 is intended for sealing connection towards the insertion tube 11 and comprises a second channel 184 provided by second body parts of the second portions. The second channel 184 provides for the insertion tube 11 to extend into the fluid joint body 180.

The fluid joint body 180 provides for a fluid passage for communicating a fluid with the interstitial space of the insertion cord 10. The first and second body part provide together in the assembled condition of the fluid joint body 180 a third channel 182d for the fluid passage to extend into the fluid joint body 180. Such fluid passage may be provided by the irrigation tube 46.

As shown, optionally, the first, second, and third channel 182a, 184, 182d each comprises an intermediate branch channel 192 extending from an intermediate position in the respective channel to an outer surface 193 of the second body part 180b.

In use of the fluid joint body 180 for assembling an endoscope, prior to assembling the first and second body parts 180a, 180b, glue may be applied to the assembly faces 190a, 190b, especially to the valleys 191a and to the parts to provide the first, second, and third channels 182a, 184, 182d. The at least one of a working channel tube 34, a pull cable 170 or a component 97, 98, the insertion tube 11, and the irrigation tube 46 may be positioned on the second body part 180b. The first body part 180a may be assembled with the second body part 180b thereby finishing the first, second, and third channels 182a, 184, 182d with at least one of a working channel tube 34, a pull cable 170 or a component 97, 98, the insertion tube 11, and the irrigation tube 46 positioned and sealed by glue. If the amount of glue provided in the respective first, second, and third channel 182a, 184, and 182d are not sufficient to effectively seal between the surface of the respective channel and the elements positioned therein, additional glue may be injected into the channels through the intermediate branch channels 192.

The fluid joint body 180 comprises an inner space 48 defined inside the fluid joint body. The inner space 48 is in fluid connection with the interstitial space of the insertion tube 11 and sealed relative to the internal space 2b of the handle housing 2a (see FIG. 3a). The first, second, and third channels 182a, 184, 182d provide for communication with the inner space 48.

For the glue and/or sealant as described herein a UV setting glue may be used, such as MD® Medical Adhesive 1187-M of the company, DYMAX, but the person skilled in the art will easily realise that many alternatives are useable.

The valleys 191a and ridges 191b provide a tongue-in-groove joint between the first body part 180a and the second body part 180b. In alternative embodiments the tonge-in-groove joint is substituted by a half-lap joint. In further alternative embodiments the joint is substituted by using adhesive to secure the first body part 180a and the second body part 180b to each other.

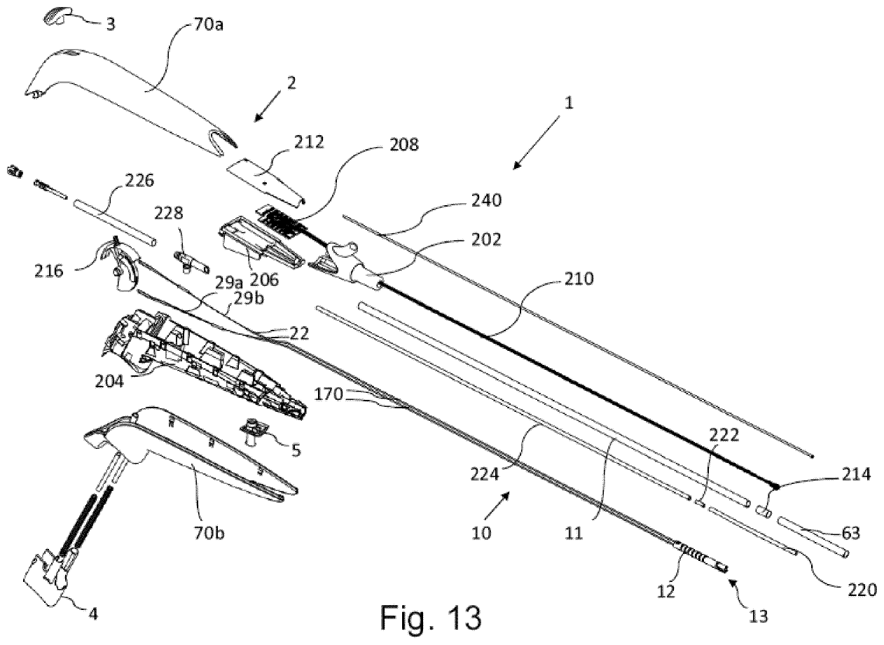

FIG. 13 shows an exploded view of the endoscope 1 comprising the handle 2 and the insertion cord 10. The handle housing 2a comprises handle portions or parts 70a and 70b and a transition part 202. Inside the handle 2, in an assembled condition, a chassis 204 is provided for supporting the parts of the handle 2. Further, inside the handle 2 is found a mounting plate 206, a printed circuit board 208 connected to the signal cables or wires 24, and a cover 212.

The insertion tube 11 is connected to the handle 2 at the transition part 202. A sleeve 214 connects the insertion tube 11 with the bending section 12 and the bending section sleeve 63. The pull cables 170 extend from the bending section 12 to the inside of the handle 2. The two pull wires 29a and 29b are attached to a control mechanism with the control lever 216. Thus, an operator may activate the bending section 12 using the control knob 3 which is mechanically attached ot the control lever 216.

The working channel 20 is provided by a first tube segment 220, located in the bending section, connected by a short length of rigid tubing 222 to a second tube segment 224. A flexible hose 226 is also shown. A T-joint 228 attached, in the assembled condition, to the connector 5. A conduit 240 is, optionally, provided surrounding the signal cables or wires 163 to extend through inside the insertion cord 10.

The following items are examples of various embodiments disclosed above:
1. An endoscope comprising a handle housing and an insertion cord, the insertion cord comprising a first tubular body and having a proximal end and a distal end, the first tubular body housing at least one of: a working channel, constituted by a second tubular body; a wire; and a wire pipe, the first tubular body comprising an interstitial lumen between any present second tubular body; wire; and wire pipe, said interstitial lumen providing a channel for flowing a fluid through the insertion cord, the handle housing comprising an internal space, wherein the proximal end of the insertion cord is attached to the handle housing, and the interstitial lumen of the first tubular body is sealed relative to the internal space of the handle housing by a sealing body, wherein said sealing body comprises at least one integral part moulded in one piece, said integral part comprising a first portion with at least a first part extending snugly along at least a part of a circumference of at least one of any second tubular body; wire; and wire pipe and a second portion abutting snugly towards the first tubular body, and wherein a fluid passage for communicating a fluid with the interstitial lumen is attached to the sealing body.
2. An endoscope according to item 1, wherein the second portion comprises a second part extending snugly along at least a part of a circumference of the first tubular body.
3. An endoscope according to item 1, wherein the sealing body comprises a number of two or more of said integral parts, whereby the first parts of the first portions of said integral parts jointly extend snugly around the entire circumferences of the at least one of any second tubular body; wire; and wire pipe.
4. An endoscope according to item 2, wherein the sealing body comprises a number of two or more of said integral parts, whereby the first parts of the first portions of said integral parts jointly extend snugly around the entire circumferences of the at least one of any second tubular body; wire; and wire pipe, and whereby the second parts of the second portions of said integral parts jointly extend snugly around the entire circumferences of the first tubular body.
5. An endoscope according to item 3 or 4, wherein the sealing body comprises a first sealing body portion comprising the joint first portions of the integral parts and a second sealing body portion comprising the joint second portions of the integral parts, and a first direction is extending from the first sealing body portion towards the second sealing body portion, and wherein the first sealing body portion is extended relative to the second sealing body portion in a second direction perpendicular to the first direction along a dividing line between said integral parts, the at least one of any second tubular body; wire; wire pipe; and fluid passage being positioned on said dividing line.
6. An endoscope according to item 5, wherein more of said at least one of any second tubular body; wire; wire pipe; and fluid passage are positioned on said dividing line and are mutually spaced whereby the integral parts on either side of the dividing line meet on the dividing line between any pair of said at least one of any second tubular body; wire; wire pipe; and fluid passage for the first parts of said integral parts jointly to extend snugly around each of said at least one of any second tubular body; wire; wire pipe; and fluid passage individually.
7. An endoscope according to item 5 or 6, wherein the first sealing body portion is extended to have a larger extent in the second direction relative to the extent of the first sealing body portion in a third direction perpendicular to the first and the second direction.
8. An endoscope according to any one of items 3 to 7, wherein the fluid passage comprises a tubular element, and wherein third parts of the first portions of said integral parts jointly extend snugly around an entire circumferences of the tubular element.
9. An endoscope according to item 1, wherein the sealing body is constituted by a single moulded body.
10. An endoscope according to item 9, wherein the single moulded body is moulded in situ to seal in relation to first tubular body; and the at least one of any second tubular body; wires; wire pipes; and the fluid passage.
11. An endoscope according to any one of the preceding items, wherein at least one wire is constituted by a Bowden cable comprising an inner cable housed in a hollow outer cable housing, whereby the hollow outer cable housing comprises a pipe sealing extending from the first part and a distance in at least one of a distal direction and a proximal direction of the Bowden cable.
12. An endoscope according to item 11, wherein a sealing glue is present in the hollow outer cable housing at the place where the hollow outer cable housing is passing the first part and a distance in at least one of the distal direction and the proximal direction of the Bowden cable, and wherein a slip means, such as oil is present between the sealing glue and the inner cable.
13. An endoscope according to item 12, wherein the pipe sealing is constituted by the sealing glue.
14. An endoscope according to any one of the preceding items, wherein the handle housing comprises a connector for connecting a fluid connection, especially a liquid connection, such as a hose, the handle housing further comprising two housing parts interconnected along respective channel interfaces extending from the connector to the sealing body at a point of attachment of the insertion cord to the handle housing, said channel interfaces providing between them a tubular channel extending along a part of the handle housing.
15. An endoscope according to any one of the preceding items, wherein the fluid passage is an irrigation liquid inlet.
16. An endoscope comprising: a handle housing comprising an internal space, a sealing body comprising a first passage and a second passage, and a fluid inlet connector adapted to receive a fluid from a fluid source; an insertion cord having a distal end and a proximal end, the proximal end attached to the handle housing, the insertion cord including: a first tubular body, a second tubular body, a wire and a wire pipe; and a fluid passage fluidly coupled to the fluid inlet connector, wherein the first tubular body comprises an interstitial lumen configured to flow a fluid through the insertion cord, the interstitial lumen surrounding at least the second tubular body, wherein the fluid passage is in fluid communication with the interstitial lumen to provide the fluid to the interstitial lumen, wherein the sealing body fluidly seals the interstitial lumen from the internal space of the handle housing, wherein the first passage receives a portion of the second tubular body, and wherein the second passage receives at least a portion of the wire or the wire pipe.
17. An endoscope according to item 16, wherein the fluid passage is comprised by a tubular element positioned in the handle housing, wherein the handle housing comprises an external wall, and wherein the external wall comprises the fluid inlet connector, the fluit inlet connector being in fluid communication with the tubular element.
18. An endoscope according to item 16, wherein the second tubular body, the wire pipe, and the wire are positioned, at least in part, within the sealing body.
19. An endoscope according to any one of the preceding items, wherein the bending section includes an interstitial space and a fluid opening, the interstitial space being in fluid communication with the interstitial lumen to receive the fluid, and wherein the fluid opening is in fluid communication with a fluid discharge opening to discharge the fluid therethrough, the fluid discharge opening comprising at least one of an hole in the bending section sleeve, a slit in the bending section sleeve, or a gap between a distal end of the bending section sleeve and the distal end segment of the bending section.
20. An endoscope system comprising: an endoscope according to any one of items 1 to 19, and a monitor.
21. An insertion cord for an endoscope, comprising a first tubular body and having a proximal end and a distal end, the first tubular body housing at least one of: a second tubular body; a wire; and a wire pipe, the first tubular body comprising an interstitial lumen between any present second tubular body; wire; and wire pipe, said interstitial lumen providing a channel for flowing a fluid through the insertion cord, the insertion cord further comprising a distal tip provided by an elongated body part comprising a circumferential body surface and a distal end tip adjacent the circumferential body surface, an outer tubular sheath providing an external surface of at least a distal part of the insertion cord adjacent the distal end tip of the elongated body part, said outer tubular sheath being at least in part exposed to the interstitial lumen, wherein said outer tubular sheath provides at least one fluid opening for fluid to pass between the interstitial lumen and a surrounding of the insertion cord.
22. An insertion cord according to item 21, wherein the at least one fluid opening is closed in a relaxed condition of the outer tubular sheath and opens when the outer tubular sheath is subject to a pressure of fluid introduced through the proximal end of the insertion cord.
23. An insertion cord according to item 21 or 22, wherein the outer tubular sheath is of a stretchable material adapted to be stretched when subject to a pressure of fluid introduced through the proximal end of the insertion cord.
24. An insertion cord according to any one of the preceding items, wherein a distal portion of the outer tubular sheath is surrounding a part of the elongated body part and is preferably attached to the circumferential body surface thereof at separate positions circumferentially space around said circumferential body surface, thus leaving the outer tubular sheath unattached to the circumferential body surface at at least one position between spaced positions of attachment.
25. An insertion cord according to item 24, wherein the outer tubular sheath, at least in a relaxed condition, surrounds the circumferential body surface with a tight fit.
26. An insertion cord according to item 24 or 25, wherein the elongated body part comprises at least one groove in the circumferential body surface, said at least one groove extending from a proximal end thereof to a distal end thereof, and said groove extending between positions of attachment of the outer tubular sheath to the circumferential body surface.
27. An insertion cord according to any one of the preceding items, wherein the at least one fluid opening is provided by a slit through the material of the outer tubular sheath.
28. An insertion cord according to any one of the preceding items, wherein the distal part comprises a bending section.
29. An insertion cord according to item 28, wherein the bending section comprises a row of segments, where adjacent segments are connected by hinges, preferably made integrally from the same material as the segments, the hinges being arranged for allowing the connected segments to bend in a bending plane, wherein the interstitial lumen extends through at least a number of the segments at a proximal end of the row of segments and the outer tubular sheath is exposed to the interstitial volume between adjacent segments.
30. An insertion cord according to item 29, wherein the elongated body part comprises a distal segment of the row of segments.
31. An insertion cord according to item 30, wherein the elongated body part includes a tip housing attached to the distal segment of the row of segments.
32. An insertion cord according to any one of the preceding items, wherein the outer tubular sheath is more flexible than the first tubular body.
33. An insertion cord according to any one of items 21-32, wherein the interstitial lumen and the at least one fluid opening are adapted for flowing irrigation liquid through the insertion cord from the proximal end thereof and exit the at least one fluid opening to the surrounding of the insertion cord.
34. An endoscope comprising a handle with an internal space, and an insertion cord according to any one of items 21-33, wherein the proximal end of the insertion cord is attached to the handle.
35. An endoscope according to item 34, wherein the interstitial lumen of the first tubular body is sealed relative to the internal space of the housing.
36. An endoscope system comprising an endoscope according to item 34 or 35, and a monitor.

1. An endoscope comprising:
a handle housing comprising an internal space, the handle comprising two interconnected housing parts;
an insertion cord having a distal end and a proximal end, the proximal end attached to the handle housing, the insertion cord including:
an insertion tube; and
at least one of a tubular body constituting a working channel, a wire and/or a wire pipe, the at least one of the tubular body, the wire and/or the wire pipe housed in the insertion tube;
a sealing body positioned in the internal space of the handle housing and comprising a sealing body internal space; and
a tubular member or a tubular channel, comprising an outlet end and a fluid passage extending to the outlet end, the tubular member positioned in the internal space of the handle, the tubular channel formed by the housing parts, the outlet end being sealingly connected to the sealing body and configured to discharge a fluid into the sealing body internal space,
wherein the insertion tube comprises a proximal end and an interstitial lumen between any present of the at least one of the tubular body, the wire and/or the wire pipe, said interstitial lumen providing a channel for flowing the fluid through the insertion cord,
wherein the proximal end of the insertion tube is sealingly connected to, and is sealed from the internal space of the handle housing by, the sealing body,
wherein the sealing body extends about the insertion tube and any present of the at least one of the tubular body, the wire and the wire pipe,
and
wherein the fluid passage establishes fluid communication with the interstitial lumen to flow the fluid to the interstitial lumen.

2. (canceled)

3. (canceled)

4. The endoscope of claim 1,
wherein the insertion cord further comprises a bending section connected to the insertion tube, a distal tip connected to the bending section, an outer tubular sheath covering the bending section, and a fluid discharge opening,
wherein the bending section includes an interstitial space and a fluid opening, the interstitial space being in fluid communication with the interstitial lumen to receive the fluid, and
wherein the fluid opening is in fluid communication with the fluid discharge opening to discharge the fluid therethrough.

5. The endoscope of claim 4, wherein the bending section comprises segments including a distal segment, a proximal segment, and intermediate segments between the distal segment and the proximal segment, and wherein the fluid opening is positioned between any two of the segments or in one of the segments.

6. The endoscope of claim 4, wherein the fluid discharge opening comprises an opening in the outer tubular sheath or an opening between the outer tubular sheath and an exterior surface of one one of the segments.

7. The endoscope of claim 4, wherein the distal tip comprises a fluid channel having a proximal end in fluid communication with the interstitial space and a distal end, the distal end comprising the fluid discharge opening.

8-21. (canceled)

22. The endoscope of claim 1, further comprising a pull wire, wherein the wire pipe and the pull wire constitute a Bowden cable, further comprising a pipe sealing extending around the wire pipe.

23. The endoscope of claim 22, wherein further comprising a sealing glue in the wire pipe and a slip means between the sealing glue and the pull wire.

24. The endoscope of claim 23, wherein the pipe sealing is constituted by the sealing glue.

25. (canceled)

26. The endoscope of claim 1, wherein the sealing body is constituted by a single moulded body, and wherein the single moulded body is moulded in situ to seal in relation to insertion tube, the at least one of the tubular body, the wire and/or the wire pipe, and the fluid passage.

27-33. (canceled)

34. The endoscope of claim 1,
wherein the insertion cord further comprises a bending section connected to the insertion tube, a distal tip connected to the bending section, an outer tubular sheath covering the bending section, and a fluid discharge opening,
wherein the bending section includes an interstitial space and a fluid opening, the interstitial space being in fluid communication with the interstitial lumen to receive the fluid, and
wherein the fluid opening is in fluid communication with the fluid discharge opening to discharge the fluid therethrough.

35. The endoscope of claim 34, wherein the bending section comprises segments including a distal segment, a proximal segment, and intermediate segments between the distal segment and the proximal segment, and wherein the fluid opening is positioned between any two of the segments or in one of the segments.

36. The endoscope of claim 34, wherein the fluid discharge opening comprises an opening in the outer tubular sheath or an opening between the outer tubular sheath and an exterior surface of one one of the segments.

37. The endoscope of claim 36, wherein the fluid discharge opening is closed in a relaxed condition of the outer tubular sheath and opens when the outer tubular sheath is subject to a pressure of the fluid introduced through the proximal end of the insertion cord.

38. The endoscope of claim 34, wherein the distal tip comprises a fluid channel having a proximal end in fluid communication with the interstitial space and a distal end, the distal end comprising the fluid discharge opening.

39. An endoscope system comprising:
the endoscope of claim 1; and
a video processing apparatus.

40-56. (canceled)

57. An endoscope comprising:
a handle including a handle housing, an irrigation inlet port adapted to receive irrigation fluid, and an irrigation channel; and
an insertion cord extending distally from the handle, the insertion cord including:
an insertion tube;
components extending through the insertion tube;
a bending section extending from the insertion tube, the bending section comprising a one-piece part comprising segments and hinges interconnecting the segments to each other, the segments including a distal segment;
a bending section cover;
a distal tip extending from the bending section and including a tip housing;
a camera at least partially enclosed in the tip housing;
interstitial space within the insertion tube in fluid communication with the irrigation channel, the interstitial space comprising space within the insertion tube not occupied by the components; and
at least one interstitial flow opening in fluid communication with the interstitial space and adapted to discharge the irrigation fluid.

58. (canceled)

59. The endoscope of claim 57, wherein the at least one interstitial flow opening comprises a slit in the bending section cover.

60. The endoscope of claim 57, wherein the bending section cover overlaps the distal segment and is bonded to the distal segment or the tip housing at circumferentially spaced bonding sites with unbonded portions of the bending section cover disposed between the bonding sites, and wherein the at least one interstitial flow opening comprises a gap between an unbonded portion of the bending section cover and the distal segment or the tip housing.

61-62. (canceled)

63. The endoscope of claim 57, wherein the tip housing comprises a distal wall and a circumferential wall extending proximally from the distal wall, and wherein the at least one interstitial flow opening comprises a passage in the circumferential wall or in the front wall.

64. (canceled)

65. The endoscope of claim 57, further comprising a component extending from the handle housing through the insertion tube and a fluid joint body inside the handle housing, the fluid joint body including:
an irrigation passage to sealingly and fluidly connect an internal space of the fluid joint body with the irrigation fluid channel;
an insertion tube passage to sealingly receive the insertion tube and thereby establish fluid communication between the internal space and the interstitial space of the insertion tube; and
component inlet and outlet passages to receive the component, the component extending through the internal space from the inlet to the outlet.

66-69. (canceled)

70. A visualization system comprising:
the endoscope of claim 57; and
a video processing apparatus operable to receive live images from the endoscope.

71. (canceled)
